(19) United States
(12) Patent Application Publication
(10) Pub. No.: US 2012/0283130 A1
(43) Pub. Date: Nov. 8, 2012

(54) METHOD FOR SCREENING INDUCED PLURIPOTENT STEM CELLS

(76) Inventors: Shinya Yamanaka, Kyoto (JP); Michiyo Koyanagi, Kyoto (JP)

(21) Appl. No.: 13/522,223

(22) PCT Filed: Jan. 17, 2011
(86) PCT No.: PCT/JP2011/051144

§ 371 Date: Jul. 13, 2012

Related U.S. Application Data
(60) Provisional application No. 61/282,295, filed on Jan. 15, 2010.

(51) Int. Cl. C12Q 1/68; C12N 5/071; C40B 40/06; C40B 30/04; C07H 21/04

(52) U.S. Cl. 506/9; 536/24.33; 536/24.31; 506/16; 435/325; 435/6.12

(57) ABSTRACT

The present invention relates to miRNA or genes expressed in induced pluripotent stem cells, and a method for screening for induced pluripotent stem cells having functions equivalent to those of embryonic stem cells by confirming methylation of specific gene regions of induced pluripotent stem cells.

### BACKGROUND OF THE INVENTION

1. Technical Field

The present invention relates to a method for screening induced pluripotent stem cells. More specifically, the present invention relates to miRNA or genes that are expressed in induced pluripotent stem cells, or a method for selecting induced pluripotent stem cells having functions equivalent to those of embryonic stem cells by confirming methylation of specific gene regions of induced pluripotent stem cells.

2. Background Art

In recent years, mouse and human induced pluripotent stem cells (iPS cells) have been successively established. Yamanaka et al., have induced iPS cells by introducing Oct3/4, Sox2, Klf4, and c-Myc genes into mouse-derived fibroblasts so as to enable the forced expression of such genes (WO 2007/069666 A1 and Takahashi, K. and Yamanaka, S., Cell, 126: 663-676 (2006)). Subsequently, it has been revealed that iPS cells can also be prepared using 3 of the above factors excluding the c-Myc gene (Nakagawa, M. et al., Nat. Biotethnol., 26: 101-106 (2008)). Furthermore, Yamanaka et al., have succeeded establishing iPS cells by introducing the 4 above genes into human skin-derived fibroblasts, similarly to the case involving mice (WO 2007/069666 A1 and Takahashi, K. et al., Cell, 131: 861-872 (2007)). Meanwhile, Thomson et al.,'s group has prepared human iPS cells using Nanog and Lin28 instead of Klf4 and c-Myc (WO 2008/118820 A2 and Yu, J. et al., Science, 318: 1917-1920 (2007)). The thus obtained iPS cells are prepared using cells from a patient to be treated, following which they can be differentiated into cells of different tissues. Thus, it is expected that iPS cells will be used as rejection-free grafting materials in the field of regenerative medicine.

However, the thus established iPS cells exert almost the same appearance and expression status of undifferentiated specific genes as those of ES cells, but the involvement in the germ line may differ from the case of ES cells (Okita K. et al., Nature, 448: 313-317 (2007)).

Also, many clones can be obtained simultaneously with the use of iPS cells, but they do not always have identical functions.

Therefore, a method for selecting iPS cells that have unlimitedly high differentiation potency, as in the case of ES cells, from among many established iPS cells has been required. However, a method that involves confirming the presence of iPS cell-derived tissue in 2^{nd}-generation mice obtained by mating iPS cell-derived chimeric mice takes a great deal of time. Also, such confirmation using human iPS cells poses a major ethical problem. Hence, it is difficult to detect whether or not established iPS cells have differentiation potency that enables germline transmission.

### SUMMARY OF INVENTION

An object of the present invention is to provide an index for conveniently screening for an induced pluripotent stem cell(s) (iPS cell(s)) having unlimitedly high differentiation potency and being capable of germline transmission. The iPS cells can be induced from somatic cells of a subject, which is an animal, preferably a mammal including humans, mice, rats, pigs, cows, and the like.

The present inventors have confirmed microRNA (hereinafter, miRNA) expression using iPS cells having various backgrounds to achieve the above object. As a result, the present inventors have found that iPS cells capable of germline transmission and iPS cells incapable of germline transmission can be distinguished based on miRNA that is expressed in the Dlk1-Dio3 region as an imprinted region. Also, the present inventors have found that, among the expression levels of genes located within the same region as that of the above miRNA, a similar correlation exists with regard to the expression levels of genes that are expressed from maternally derived chromosomes. Thus, they have confirmed that such miRNA can be used as an index for screening for iPS cells in which germline transmission occurs. They have also found that iPS cells can be similarly screened for by confirming DNA methylation in a region that controls the expression of genes of the Dlk1-Dio3 region.

Based on the above results, the present inventors have found that iPS cells having unlimitedly high differentiation potency and being capable of germline transmission as in the case of ES cells can be selected by detecting miRNA or the gene of imprinted region or DNA methylation in imprinted region. Thus, they have completed the present invention.

The present invention is as follows.

[1] A method for screening an induced pluripotent stem cell(s), comprising the following steps of:
(1) measuring the expression level of at least one miRNA or gene located in an imprinted region in a subject induced pluripotent stem cell(s); and,
(2) selecting the induced pluripotent stem cell(s) expressing the miRNA or the gene at a level equivalent to or higher than that of a control cell(s).

[2] The method according to [1], wherein the imprinted region is a Dlk1-Dio3 region.

[3] The method according to [1], wherein the miRNA is selected from the group consisting of the pri-miRNA shown in Tables 1 and 3 and the mature-miRNA shown in Tables 2 and 4.

[4] The method according to [1], wherein the gene is selected from the group consisting of genes shown in Table 5.

[5] The method according to [4], wherein the gene is selected from the group consisting of MEG3 and MEG8.

[6] The method according to [1], wherein the control cell(s) is/are an embryonic stem cell(s).

[7] A method for screening induced pluripotent stem cells, comprising the following steps of:
(1) measuring a DNA methylation state in an imprinted region of a subject induced pluripotent stem cell(s); and
(2) selecting the induced pluripotent stem cell(s) in which the imprinted region in a/one chromosome is in a DNA-methylated state, but the same region in a homologous chromosome is not in a DNA-methylated state.

[8] The method according to [7], wherein the imprinted region is IG-DMR and/or Gtl2/MEG3-DMR.

[9] The method according to [7], comprising the step of selecting an induced pluripotent stem cell(s) in which the imprinted region in a paternally-derived chromosome is in the DNA-methylated state.

[10] The method according to [1] or [9], wherein the induced pluripotent stem cell(s) is/are capable of germline transmission.

[11] A kit for screening induced pluripotent stem cells, which comprises at least one primer set or probe for detecting pri-miRNA shown in Table 1 or 3, miRNA shown in Table 2 or 4, and a gene shown in Table 5.

[12] The kit according to [11], which comprises a microarray.

[13] A kit for screening induced pluripotent stem cells, which comprises a methylation-sensitive restriction enzyme, or a bisulfite reagent and a nucleic acid for amplification of IG-DMR and/or Gtl2/MEG3-DMR.

[14] An induced pluripotent stem cell capable of germline transmission, which is screened for by the method according to any one of [1] to [10].

### BRIEF DESCRIPTION OF DRAWINGS

FIG. 1 shows the results of hierarchical clustering of microarray data of miRNA expressed in ES cells, iPS cells, and somatic cells. Here, values within the color range are log 2 values of detected signal intensity. Red indicates strong expression signals and blue indicates weak expression signals. Group I is a group specifically expressed in ES cells and iPS cells. Group II is a group expressed non-specifically among iPS cells.

FIG. 2 shows the results of detailed microarray analyses for miRNA (A) of Group I and miRNA (B) of Group II in ES cells, iPS cells, and somatic cells. The clone name of each cell is shown in the lower area and the ID names of miRNA are shown in the area on the right. Here, values in the color range are log 2 values of detected signal intensity. Red indicates strong expression signals and blue indicates weak expression signals.

FIG. 3 is a schematic diagram showing locations of miRNA and genes in human and mouse Dlk1-Dio3 regions.

FIG. 4 shows the results of microarray analyses by which the expression of genes located in the Dlk1-Dio3 region in ES cells, iPS cells, and somatic cells was examined. The clone name of each cell is shown in the lower area and gene names are shown in the right area. Results are normalized by the Quantile normalization method and expressed by signal intensity. Here, Red indicates strong expression signals and blue indicates weak expression signals.

FIG. 5 shows the results of measuring the methylation state of CG sequences at 17 positions in IG-DMR of ES cells (RF8) and iPS cells (178B5 and 335D3) by the Bisulfite method. A filled circle indicates that the CG sequence was methylated and an open circle indicates that the CG sequence was not methylated. The measurement results shown in FIG. 5 were: 61 clones for RF8, 54 clones for 178B5, and 53 clones for 335D3.

FIG. 6 shows the results of microarray analyses by which the expression of miRNA located in the DLK1-DIO3 region in human ES cells, human iPS cells, and human somatic cells was examined. The clone name of each cell is shown in the lower area and miRNA names are shown in the right area. Results are normalized by the Quantile normalization method and expressed by signal intensity. Here, Red indicates strong expression signals and blue indicates weak expression signals.

FIG. 7 shows the results of microarray analyses by which the expression of miRNA located in the DLK1-DIO3 region in human ES cells and human iPS cells was examined. The clone name is shown in the lower area and miRNA names are shown in the right area. The number following clone name means passage number. Results are normalized by the Quantile normalization method and expressed by signal intensity. Here, Red indicates strong expression signals and green indicates weak expression signals.

FIG. 8 shows the results of expression level of MEG3 (gray-bar) and MEG8 (black-bar) in each cell line measuring with quantitative PCR. The clone name is shown in the lower area. The expression level of KhES1 is used as standard and each level is normalized with GAPDH expression level.

FIG. 9 is a schematic diagram showing locations of IG-DMR CG4, MEG3-DMR CG7 and relating genes.

FIG. 10 shows the results of measuring the methylation state of CG sequences in IG-DMR CG4 and MEG3-DMR CG7 of 3 clones of ES cells (KhES1, KhES3 and H1) and 3 clone of iPS cells (DP31-4F1, 201B7 and 201B6) by the Bisulfite method. There are 8 CG positions (indicating “A”) and 9 CG positions (indicating “G”), because of SNP (A/G) in IG-DMR CG4 region. A filled circle indicates that the CG sequence was methylated and an open circle indicates that the CG sequence was not methylated.

### MODES FOR CARRYING OUT THE INVENTION

The present invention provides a method and a kit for screening for induced pluripotent stem cells (iPS cells) having unlimitedly high differentiation potency and being capable of germline transmission.

### Method for Producing iPS Cells

Induced pluripotent stem (iPS) cells can be prepared by introducing a specific nuclear reprogramming substance in the form of DNA or protein into somatic cells. iPS cells are somatic cell-derived artificial stem cells having properties almost equivalent to those of ES cells, such as pluripotency and proliferation potency via self-renewal (K. Takahashi and S. Yamanaka (2006) Cell, 126: 663-676; K. Takahashi et al. (2007) Cell, 131: 861-872; J. Yu et al. (2007) Science, 318: 1917-1920; M. Nakagawa et al. (2008) Nat. Biotechnol., 26: 101-106; international publication WO 2007/069666). A nuclear reprogramming substance may be a gene specifically expressed in ES cells, a gene playing an important role in maintenance of undifferentiation of ES cells, or a gene product thereof. Examples of such nuclear reprogramming substance include, but are not particularly limited to, Oct3/4, Klf4, Klf1, Klf2, Klf5, Sox2, Sox1, Sox3, Sox15, Sox17, Sox18, c-Myc, L-Myc, N-Myc, TERT, SV40 Large T antigen, HPV16 E6, HPV16 E7, Bmi1, Lin28, Lin28b, Nanog, Esrrb, and Esrrg. These reprogramming substances may be used in combination upon establishment of iPS cells. Such combination may contain at least one, two, or three reprogramming substances above and preferably contains 4 reprogramming substances above.

The nucleotide sequence information of the mouse or human cDNA of each of the above nuclear reprogramming substances and the amino acid sequence information of a protein encoded by the cDNA can be obtained by referring to NCBI accession numbers described in WO 2007/069666. Also, the mouse and human cDNA sequence and amino acid sequence information of L-Myc, Lin28, Lin28b, Esrrb, and Esrrg can be each obtained by referring to the following NCBI accession numbers. Persons skilled in the art can prepare desired nuclear reprogramming substances by a conventional technique based on the cDNA sequence or amino acid sequence information.

| Gene name | Mouse | Human |
|---|---|---|
| L-Myc | NM_008506 | NM_001033081 |
| Lin28 | NM_145833 | NM_024674 |
| Lin28b | NM_001031772 | NM_001004317 |
| Esrrb | NM_011934 | NM_004452 |
| Esrrg | NM_011935 | NM_001438 |

These nuclear reprogramming substances may be introduced in the form of protein or mature mRNA into somatic cells by a technique such as lipofection, binding with a cell membrane-permeable peptide, or microinjection. Alternatively, they can also be introduced in the form of DNA into somatic cells by a technique such as a technique using a vector such as a virus, a plasmid, or an artificial chromosome, lipofection, a technique using a liposome, or microinjection. Examples of a viral vector include a retrovirus vector, a lentivirus vector (these are according to Cell, 126, pp. 663-676, 2006; Cell, 131, pp. 861-872, 2007; and Science, 318, pp. 1917-1920, 2007), an adenovirus vector (Science, 322, 945-949, 2008), an adeno-associated virus vector, and a Sendai virus vector (Proc Jpn Acad Ser B Phys Biol Sci. 85, 348-62, 2009). Also, examples of an artificial chromosome vector include a human artificial chromosome (HAC), a yeast artificial chromosome (YAC), and a bacterial artificial chromosome (BAC and PAC). As a plasmid, a plasmid for mammalian cells can be used (Science, 322: 949-953, 2008). A vector can contain regulatory sequences such as a promoter, an enhancer, a ribosome binding sequence, a terminator, and a polyadenylation site, so that a nuclear reprogramming substance can be expressed. A vector may further contain, if necessary, a selection marker sequence such as a drug resistant gene (e.g., a neomycin resistant gene, an ampicillin resistant gene, and a puromycin resistant gene), a thymidine kinase gene, and a diphtheria toxin gene, and a reporter gene sequence such as a green fluorescent protein (GFP), β glucuronidase (GUS), and FLAG. Also, in order to cleave both a gene encoding a nuclear reprogramming substance or a promoter and a gene encoding a nuclear reprogramming substance binding thereto after introduction into somatic cells, the above vector may have LoxP sequences located before and after the relevant portion. Furthermore, the above vector may also contain EBNA-1 and oriP, or Large T and SV40ori sequences so that they can be episomally present and replicated without incorporation into a chromosome.

Upon nuclear reprogramming, to improve the efficiency for inducing iPS cells, in addition to the above factors, histone deacetylase (HDAC) inhibitors [e.g., low-molecular weight inhibitors such as valproic acid (VPA) (Nat. Biotechnol., 26(7): 795-797 (2008)), trichostatin A, sodium butyrate, MC 1293, and M344, and nucleic acid expression inhibitors such as siRNA and shRNA against HDAC (e.g., HDAC1 siRNA Smartpool® (Millipore) and HuSH 29mer shRNA Constructs against HDAC1 (OriGene))], DNA methyltransferase inhibitors (e.g., 5′-azacytidine) (Nat. Biotechnol., 26(7): 795-797 (2008)), G9a histone methyltransferase inhibitors [e.g., low-molecular-weight inhibitors such as BIX-01294 (Cell Stem Cell, 2: 525-528 (2008)) and nucleic acid expression inhibitors such as siRNA and shRNA against G9a (e.g., G9a siRNA (human) (Santa Cruz Biotechnology))], L-channel calcium agonists (e.g., Bayk8644) (Cell Stem Cell, 3, 568-574 (2008)), p53 inhibitors (e.g., siRNA and shRNA against p53) (Cell Stem Cell, 3, 475-479 (2008)), Wnt Signaling (e.g., soluble Wnt3a) (Cell Stem Cell, 3, 132-135 (2008)), cytokines such as LIF or bFGF, ALK5 inhibitors (e.g., SB431542) (Nat Methods, 6: 805-8 (2009)), mitogen-activated protein kinase signaling inhibitors, glycogen synthase kinase-3inhibitors (PloS Biology, 6(10), 2237-2247 (2008)), miRNA such as miR-291-3p, miR-294, and miR-295 (R. L. Judson et al., Nat. Biotech., 27: 459-461 (2009)), for example, can be used.

Examples of a culture medium for inducing iPS cells include, but are not limited to, (1) a DMEM, DMEM/F12, or DME medium containing 10-15% FBS (these media may further appropriately contain LIF, penicillin/streptomycin, puromycin, L-glutamine, nonessential amino acids, Beta-mercaptoethanol, and the like), (2) a medium for ES cell culture containing bFGF or SCF, such as a medium for mouse ES cell culture (e.g., TX-WES medium (Thromb-X)), and a medium for primate ES cell culture (e.g., a medium for primate (human &monkey) ES cells, ReproCELL, Kyoto, Japan).

An example of culture methods is as follows. Somatic cells are brought into contact with nuclear reprogramming substances (DNA or protein) on a DMEM or DMEM/F12 medium containing 10% FBS at 37° C. in the presence of 5% CO₂ and are cultured for about 4 to 7 days. Subsequently, the cells are reseeded on feeder cells (e.g., mitomycin C-treated STO cells or SNL cells). About 10 days after contact between the somatic cells and the nuclear reprogramming factors, cells are cultured in a bFGF-containing medium for primate ES cell culture. About 30-45 days or more after the contact, iPS cell-like colonies can be formed. Cells may also be cultured under conditions in which the oxygen concentration is as low as 5%-10% in order to increase the efficiency for inducing iPS cells.

Alternatively, cells may be cultured using a DMEM medium containing 10% FBS (which may further appropriately contain LIF, penicillin/streptomycin, L-glutamine, nonessential amino acids, beta-mercaptoethanol, and the like) on feeder cells (e.g., mitomycin C-treated STO cells or SNL cells). After about 25-30 days or more, ES cell-like colonies can be formed.

During the above culture, medium exchange with fresh medium is preferably performed once a day from day 2 after the start of culture. In addition, the number of somatic cells to be used for nuclear reprogramming is not limited, but ranges from approximately 5×10³ to approximately 5×10⁶ cells per culture dish (100 cm²).

When a gene containing a drug resistant gene is used as a marker gene, cells expressing the marker gene can be selected by culturing the cells in a medium (selective medium) containing the relevant drug. Also, cells expressing the marker gene can be detected when the marker gene is a fluorescent protein gene, through observation with a fluorescence microscope, by adding a luminescent substrate in the case of a luminescent enzyme gene, or adding a chromogenic substrate in the case of a chromogenic enzyme gene.

The term “somatic cells” as used herein may refer to any cells other than germ cells from mammals (e.g., humans, mice, monkeys, pigs, and rats). Examples of such somatic cells include keratinizing epithelial cells (e.g., keratinizing epidermal cells), mucosal epithelial cells (e.g., epithelial cells of the surface layer of tongue), exocrine epithelial cells (e.g., mammary glandular cells), hormone-secreting cells (e.g., adrenal medullary cells), cells for metabolism and storage (e.g., hepatocytes), boundary-forming luminal epithelial cells (e.g., type I alveolar cells), luminal epithelial cells of internal tubules (e.g., vascular endothelial cells), ciliated cells having carrying capacity (e.g., airway epithelial cells), cells for secretion to extracellular matrix (e.g., fibroblasts), contractile cells (e.g., smooth muscle cells), cells of blood and immune system (e.g., T lymphocytes), cells involved in sensation (e.g., rod cells), autonomic nervous system neurons (e.g., cholinergic neurons), sense organ and peripheral neuron supporting cells (e.g., satellite cells), nerve cells and glial cells of the central nervous system (e.g., astroglial cells), chromocytes (e.g., retinal pigment epithelial cells), and precursor cells thereof (tissue precursor cells). Without particular limination concerning the degree of cell differentiation, the age of an animal from which cells are collected, or the like, both undifferentiated precursor cells (also including somatic stem cells) and terminally-differentiated mature cells can be similarly used as origins for somatic cells in the present invention. Examples of undifferentiated precursor cells include tissue stem cells (somatic stem cells) such as neural stem cells, hematopoietic stem cells, mesenchymal stem cells, and dental pulp stem cells.

In the present invention, individual mammals from which somatic cells are collected are not particularly limited but are preferably humans.

### Method for Screening iPS Cells

The above-established iPS cells are subjected to detection of the expression of miRNA in at least one imprinted region or a gene to be expressed from a maternally derived chromosome from among genes located in such at least one imprinting region, or, DNA methylation in a region controlling expression of the gene located in an imprinted region. Thus, iPS cells having unlimitedly high differentiation potency and being capable of germline transmission can be selected. In the present invention, the term “imprinted region” refers to a region encoding a gene that is selectively expressed from either maternally- or paternally-derived chromosome. An example of preferable imprinted region is the Dlk1-Dio3 region.

The term “miRNA” as used herein refers to “pri-miRNA”, “pre-miRNA” and “mature-miRNA”, which concerns regulation of gene expression via inhibition of translation from mRNA to protein or mRNA degradation. The “pri-miRNA” is single strand RNA which transcribed from DNA and has a hairpin loop structure containing miRNA and its complementary strand. The “pre-miRNA” is produced from pri-miRNA partially cleaving by an intranuclear enzyme called Drosha. The “mature-miRNA” is single strand RNA (20-25 nucleotides) which is produced from pre-miRNA cleaving by Dicer outside the nucleus. Therefore, miRNA to be detected in the present invention is not limited to any of these forms including pri-miRNA, pre-miRNA, and mature-miRNA.

miRNA preferable in the present invention is miRNA transcribed from chromosome 12 in the case of mice and from chromosome 14 in the case of humans and is more preferably, miRNA located in Dlk1-Dio3 region. Further preferably, in the case of mice, preferable examples of pri-miRNA and mature-miRNA are respectively shown in Table 1 and Table 2. In the case of humans, preferable examples of pri-miRNA and mature-miRNA are respectively shown in Table 3 and Table 4. It goes without saying that miRNA to be detected herein can be appropriately selected by persons skilled in the art depending on animal species.

Examples of a method for detecting the above miRNA include, but are not particularly limited to, Northern blotting, hybridization such as in situ hybridization, an RNase protection assay, a PCR method, a real-time PCR method, and a microarray method.

A preferable detection method involves: the use of hybridization of either miRNA, which is/includes pri-miRNA and/or mature miRNA such as those listed in Tables 1 and 3 or Tables 2 and 4 (see below), or a gene such as that listed in Table 5 (see below), with a nucleic acid, which is capable of hybridizing with the miRNA or the gene, as a probe; or the use of a PCR method with primers, which are capable of amplifying a sequence of DNA encoding the miRNA or a sequence of the gene. According to the present invention, the miRNA or the gene is located in an imprinted region, preferably the Dlk1-Dio3 region, of an induced pluripotent stem cell. Preferably, the gene is MEG3 or MEG8.

Examples of the probe or primer nucleic acid include the whole or partial sequences of the RNA listed in Tables 1, 2, 3, and 4 or cDNA encoding the RNA, or the whole or partial sequences of the genes listed in Table 5 or cDNA thereof, or sequences complementary to said whole or partial sequences. The size of the probe is generally at least 15 nucleotides, preferably at least 20 nucleotides, for example 20-30 nucleotides, 30-70 nucleotides, 70-100 nucleotide or more, etc. The size of the primer is generally 17-30 or more, preferably 20-25. The synthesis of the probe or primer can be conducted chemically using a commercially available automated nucleic acid synthesizer, for example.

The probe also may be an artificial nucleic acid, such as LNA (locked nucleic acid) (this is also referred to as bridged nucleic acid (BNA)) or PNA (peptide nucleic acid), serving as an alternate for RNA having a sequence complementary to the nucleotide sequence of miRNA.

LNA has a cross-linked structure in which position 2′ and position 4′ of RNA ribose are covalently bound via methylene groups (A. A. Koshkin et al., Tetrahedron, 54: 3607 (1998); S. Obika et al., Tetrahedron Lett., 39: 5401 (1998)). PNA lacks ribose, but has a structure containing amide and ethylene imine bonds in the backbone. PNA is as described in P. E. Nielsen et al., Science 254: 1497 (1991), P. E. Nielsen ed., Peptide Nucleic Acids Protocols and Applications, 2nd ed. Horizon Bioscience (UK) (2004), for example. miRNA to be detected and an artificial nucleic acid probe hybridizable thereto such as LNA and PNA bind onto carriers on a microarray or the like, so that a large number of miRNAs can be detected and quantitatively determined simultaneously. The size of an artificial nucleic acid may range from about 10 mer to 25 mer.

If necessary, the probe as described above may be labeled. As a label, a fluorescent label (e.g., cyan, fluorescamine, rhodamine, and a derivative thereof, such as Cy3, Cy5, FITC, and TRITC) can be used.

The number of miRNA to be detected may be any number and is at least 1, at least 5, at least 10, at least 20, at least 30, at least 40 or at least 50. More preferably the number of such miRNA is 36.

TABLE 1
Pri-miRNA of mouse Dlk1-Dio3 region

| | | | SEQ |
|---|---|---|---|
| | | | ID |
| ID | Accession | Sequence | NO: |
| mmu-mir- | MI0004203 | GCCACCUUCUGUGCCCCCAGCACCACGU | 1 |
| 770 | | GUCUGGGCCACGUGAGCAACGCCACGUG | |
| | | GGCCUGACGUGGAGCUGGGGCCGCAGGG | |
| | | GUCUGAUGGC | |
| mmu-mir- | MI0004601 | UGGAGCCUGAGGGGCUCACAGCUCUGGU | 2 |
| 673 | | CCUUGGAGCUCCAGAGAAAAUGUUGCUC | |
| | | CGGGGCUGAGUUCUGUGCACCCCCCUUG | |
| | | CCCUCCA | |
| mmu-mir- | MI0005514 | CGCCAGGGCCUUGUACAUGGUAGGCUUU | 3 |
| 493 | | CAUUCAUUUUUUGCACAUUCGGUGAAGG | |
| | | UCCUACUGUGUGCCAGGCCCUGUGCCA | |
| mmu-mir- | MI0000615 | CAGUGUAGUGAGAAGUUGGGGGGUGGGA | 4 |
| 337 | | ACGGCGUCAUGCAGGAGUUGAUUGCACA | |
| | | GCCAUUCAGCUCCUAUAUGAUGCCUUUC | |
| | | UUCACCCCCUUCA | |
| mmu-mir- | MI0003518 | UGGGCCAAGGGUCACCCUCUGACUCUGU | 5 |
| 540 | | GGCCAAGGGUAGACAGGUCAGAGGUCGA | |
| | | UCCUGGGCCUA | |
| mmu-mir- | MI0004171 | AGAACAGGGUCUCCUUGAGGGGCCUCUG | 6 |
| 665 | | CCUCUAUCCAGGAUUAUGUUUUUAUGAC | |
| | | CAGGAGGCUGAGGUCCCUUACAGGCGGC | |
| | | CUCUUACUCU | |
| mmu-mir- | MI0001524 | CGUCCUGCGAGGUGUCUUGCAGGCCGUC | 7 |
| 431 | | AUGCAGGCCACACUGACGGUAACGUUGC | |
| | | AGGUCGUCUUGCAGGGCUUCUCGCAAGA | |
| | | CGACAUC | |
| mmu-mir- | MI0001525 | UGCCCGGGGAGAAGUACGGUGAGCCUGU | 8 |
| 433 | | CAUUAUUCAGAGAGGCUAGAUCCUCUGU | |
| | | GUUGAGAAGGAUCAUGAUGGGCUCCUCG | |
| | | GUGUUCUCCAGGUAGCGGCACCACACCA | |
| | | UGAAGGCAGCCC | |
| mmu-mir- | MI0000154 | CCAGCCUGCUGAAGCUCAGAGGGCUCUG | 9 |
| 127 | | AUUCAGAAAGAUCAUCGGAUCCGUCUGA | |
| | | GCUUGGCUGGUCGG | |
| mmu-mir- | MI0001526 | UCGACUCUGGGUUUGAACCAAAGCUCGA | 10 |
| 434 | | CUCAUGGUUUGAACCAUUACUUAAUUCG | |
| | | UGGUUUGAACCAUCACUCGACUCCUGGU | |
| | | UCGAACCAUC | |
| mmu-mir- | MI0012528 | UGGGUAGCUCUUGCAUUUCCUGGUGGGG | 11 |
| 432 | | GCCACUGGAUGGCUCCUCCACUUCUUGG | |
| | | AGUAGAUCAGUGGGCAGCU | |
| mmu-mir- | MI0000162 | GAGGACUCCAUUUGUUUUGAUGAUGGAU | 12 |
| 136 | | UCUUAAGCUCCAUCAUCGUCUCAAAUGA | |
| | | GUCUUC | |
| mmu-miR- | MI0000625 | AAAAUGAUGAUGUCAGUUGGCCGGUCGG | 13 |
| 341 | | CCGAUCGCUCGGUCUGUCAGUCAGUCGG | |
| | | UCGGUCGAUCGGUCGGUCGGUCAGUCGG | |
| | | CUUCCUGUCUUC | |
| mmu-mir- | MI0006290 | AUACUCACAGUCUCCCAGCUGGUGUGAG | 14 |
| 1188 | | GUUGGGCCAGGAUGAAACCCAAGGCUCU | |
| | | CCGAGGCUCCCCACCACACCCUGCUGCU | |
| | | GAAGACUGCCUAGCAAGGCUGUGCCGAG | |
| | | UGGUGUGG | |
| mmu-mir- | MI0001165 | AGACGGAGAGACCAGGUCACGUCUCUGC | 15 |
| 370 | | AGUUACACAGCUCAUGAGUGCCUGCUGG | |
| | | GGUGGAACCUGGUUUGUCUGUCU | |
| mmu-mir- | MI0005475 | CAGCAGUACCAGGAGAGAGUUAGCGCAU | 16 |
| 882 | | UAGUGCAAUAGUUAGUCCUGAUUUCUGG | |
| | | GUUUUUCUAAUGGCUGCUCUU | |
| mmu-mir- | MI0000796 | AGAGAUGGUAGACUAUGGAACGUAGGCG | 17 |
| 379 | | UUAUGUUUUUGACCUAUGUAACAUGGUC | |
| | | CACUAACUCU | |
| mmu-mir- | MI0001163 | UGGUACUUGGAGAGAUAGUAGACCGUAU | 18 |
| 411 | | AGCGUACGCUUUAUCUGUGACGUAUGUA | |
| | | ACACGGUCCACUAACCCUCAGUAUCA | |
| mmu-mir- | MI0000399 | AAGAAAUGGUUUACCGUCCCACAUACAU | 19 |
| 299 | | UUUGAGUAUGUAUGUGGGACGGUAAACC | |
| | | GCUUCUU | |
| mmu-mir- | MI0000797 | AAGAUGGUUGACCAUAGAACAUGCGCUA | 20 |
| 380 | | CUUCUGUGUCGUAUGUAGUAUGGUCCAC | |
| | | AUCUU | |
| mmu-mir- | MI0006305 | GUGAGCUGGAAUCAGCCAGCGUUACCUC | 21 |
| 1197 | | AAGGUAUUUGAAGAUGCGGUUGACCAUG | |
| | | GUGUGUACGCUUUAUUUAUGACGUAGGA | |
| | | CACAUGGUCUACUUCUUCUCAAUAUCAC | |
| | | AUCUCGCC | |
| mmu-mir- | MI0000592 | UUGGUACUUGGAGAGAGGUGGUCCGUGG | 22 |
| 323 | | CGCGUUCGCUUCAUUUAUGGCGCACAUU | |
| | | ACACGGUCGACCUCUUUGCGGUAUCUAA | |
| | | UC | |
| mmu-mir- | MI0004129 | UGGGUGCGUGAGGUGGUUGACCAGAGAG | 23 |
| 758 | | CACACGCUAUAUUUGUGCCGUUUGUGAC | |
| | | CUGGUCCACUAACCCUCAGUAUCUA | |
| mmu-mir- | MI0000605 | UGUUCGCUUCUGGUACCGGAAGAGAGGU | 24 |
| 329 | | UUUCUGGGUCUCUGUUUCUUUGAUGAGA | |
| | | AUGAAACACACCCAGCUAACCUUUUUUU | |
| | | CAGUAUCAAAUCC | |
| mmu-mir- | MI0003532 | UUGAUACUUGAAGGAGAGGUUGUCCGUG | 25 |
| 494 | | UUGUCUUCUCUUUAUUUAUGAUGAAACA | |
| | | UACACGGGAAACCUCUUUUUUAGUAUCA | |
| | | A | |
| mmu-mir- | MI0004638 | CUAUGGCUUUGGACUGUGAGGUGACUCU | 26 |
| 679 | | UGGUGUGUGAUGGCUUUUCAGCAAGGUC | |
| | | CUCCUCACAGUAGCUAUA | |
| mmu-mir- | MI0006298 | CUGAAGGGACAAUGAUGCCCACUGUUCU | 27 |
| 1193 | | CGGGGUAGCUGUGUGGAUGGUAGACCGG | |
| | | UGACGUACACUUCAUUUAUGCUGUAGGU | |
| | | CACCCGUUUUACUAUCCACCAACACCCA | |
| | | GACCAUCUG | |
| mmu-mir- | MI0004553 | CUGAUUCUGCCUGCGUGGAGCGGGCACA | 28 |
| 666 | | GCUGUGAGAGCCCCCUAGGUACAGCGGG | |
| | | GCUGCAGCGUGAUCGCCUGCUCACGCAC | |
| | | AGGAAGUGACGACAG | |
| mmu-mir- | MI0003519 | UGCUUAAUGAGAAGUUGCCCGCGUGUUU | 29 |
| 543 | | UUCGCUUUAUAUGUGACGAAACAUUCGC | |
| | | GGUGCACUUCUUUUUCAGCA | |
| mmu-mir- | MI0004639 | AAAGAAGUUGCCCAUGUUAUUUUUCGCU | 30 |
| 495 | | UUUAUUUGUGACGAAACAAACAUGGUGC | |
| | | ACUUCUU | |
| mmu-mir- | MI0004196 | GUGGGUACUGGCCUCGGUGCUGGUGGAG | 31 |
| 667 | | CAGUGAGCACGCCAUACAUUAUAUCUGU | |
| | | GACACCUGCCACCCAGCCCAAGGCCCCU | |
| | | AGGCCCAC | |
| mmu-mir- | MI0003533 | UUUGGUAUUUAAAAGGUGGAUAUUCCUU | 32 |
| 376c | | CUAUGUUUAUGCUUUUUGUGAUUAAACA | |
| | | UAGAGGAAAUUUCACGUUUUCAGUGUCA | |
| | | AA | |
| mmu-mir- | MI0005520 | CUCGGUAAGUGGGAAGAUGGUAAGCUGC | 33 |
| 654 | | AGAACAUGUGUGUUUCUCAUGUCAUAUG | |
| | | UCUGCUGACCAUCACCUUUGGGUCUCUG | |
| mmu-mir- | MI0001162 | UGGUAUUUAAAAGGUGGAUAUUCCUUCU | 34 |
| 376b | | AUGGUUACGUGCUUCCUGGAUAAUCAUA | |
| | | GAGGAACAUCCACUUUUUCAGUAUCA | |
| mmu-mir- | MI0000793 | UAAAAGGUAGAUUCUCCUUCUAUGAGUA | 35 |
| 376a | | CAAUAUUAAUGACUAAUCGUAGAGGAAA | |
| | | AUCCACGUUUUC | |
| mmu-mir- | MI0000400 | GCUACUUGAAGAGAGGUUAUCCUUUGUG | 36 |
| 300 | | UGUUUGCUUUACGCGAAAUGAAUAUGCA | |
| | | AGGGCAAGCUCUCUUCGAGGAGC | |
| mmu-mir- | MI0000798 | UACUUAAAGCGAGGUUGCCCUUUGUAUA | 37 |
| 381 | | UUCGGUUUAUUGACAUGGAAUAUACAAG | |
| | | GGCAAGCUCUCUGUGAGUA | |
| mmu-mir- | MI0003534 | UGGUACUUGGAGAGUGGUUAUCCCUGUC | 38 |
| 487b | | CUCUUCGCUUCACUCAUGCCGAAUCGUA | |
| | | CAGGGUCAUCCACUUUUUCAGUAUCA | |
| mmu-mir- | MI0003520 | UACUUGAGGAGAAAUUAUCCUUGGUGUG | 39 |
| 539 | | UUGGCUCUUUUGGAUGAAUCAUACAAGG | |
| | | AUAAUUUCUUUUUGAGUA | |
| mmu-mir- | MI0005555 | CACCUAGGGAUCUUGUUAAAAAGCAGAG | 40 |
| 544 | | UCUGAUUGAGGGGCCAAGAUUCUGCAUU | |
| | | UUUAGCAAGCUCUCAAGUGAUG | |
| mmu-mir- | MI0000799 | UACUUGAAGAGAAGUUGUUCGUGGUGGA | 41 |
| 382 | | UUCGCUUUACUUGUGACGAAUCAUUCAC | |
| | | GGACAACACUUUUUUCAGUA | |
| mmu-mir- | MI0000160 | AGGGUGUGUGACUGGUUGACCAGAGGGG | 42 |
| 134 | | CGUGCACUCUGUUCACCCUGUGGGCCAC | |
| | | CUAGUCACCAACCCU | |
| mmu-mir- | MI0004134 | GGUAAGUGUGCCUCGGGUGAGCAUGCAC | 43 |
| 668 | | UUAAUGUAGGUGUAUGUCACUCGGCUCG | |
| | | GCCCACUACC | |
| mmu-mir- | MI0003492 | ACUUGGAGAGAGGCUGGCCGUGAUGAAU | 44 |
| 485 | | UCGAUUCAUCUAAACGAGUCAUACACGG | |
| | | CUCUCCUCUCUUCUAGU | |
| mmu-mir- | MI0005497 | AGAAGAUGCAGGAGUGCUGUGAGAAGUG | 45 |
| 453 | | CCAUCCCCUGGUACUUGGAGGGAGGUUG | |
| | | CCUCAUAGUGAGCUUGCAUUAUUUAA | |
| mmu-mir- | MI0000176 | GAAGAUAGGUUAUCCGUGUUGCCUUCGC | 46 |
| 154 | | UUUAUUCGUGACGAAUCAUACACGGUUG | |
| | | ACCUAUUUUU | |
| mmu-mir- | MI0004589 | AGUGUUCGAAUGGAGGUUGCCCAUGGUG | 47 |
| 496 | | UGUUCAUUUUAUUUAUGAUGAGUAUUAC | |
| | | AUGGCCAAUCUCCUUUCGGCACU | |
| mmu-mir- | MI0000794 | UGAGCAGAGGUUGCCCUUGGUGAAUUCG | 48 |
| 377 | | CUUUAUUGAUGUUGAAUCACACAAAGGC | |
| | | AACUUUUGUUUG | |
| mmu-mir- | MI0003521 | GCCAAAAUCAGAGAAGGGAUUCUGAUGU | 49 |
| 541 | | UGGUCACACUCCAAGAGUUUUAAAAUGA | |
| | | GUGGCGAACACAGAAUCCAUACUCUGCU | |
| | | UAUGGC | |
| mmu-mir- | MI0001160 | UGGUACUCGGAGAGAGGUUACCCGAGCA | 50 |
| 409 | | ACUUUGCAUCUGGAGGACGAAUGUUGCU | |
| | | CGGUGAACCCCUUUUCGGUAUCA | |
| mmu-mir- | MI0001164 | GGGUAUGGGACGGAUGGUCGACCAGCUG | 51 |
| 412 | | GAAAGUAAUUGUUUCUAAUGUACUUCAC | |
| | | CUGGUCCACUAGCCGUCGGUGCCC | |
| mmu-mir- | MI0003535 | GGUACUUGAAGGGAGAUCGACCGUGUUA | 52 |
| 369 | | UAUUCGCUUGGCUGACUUCGAAUAAUAC | |
| | | AUGGUUGAUCUUUUCUCAGUAUC | |
| mmu-mir- | MI0001161 | GGGUACUUGAGGAGAGGUUGUCUGUGAU | 53 |
| 410 | | GAGUUCGCUUUAUUAAUGACGAAUAUAA | |
| | | CACAGAUGGCCUGUUUUCAAUACCA | |

TABLE 2
Mature-miRNA of mouse Dlk1-Dio3 region

| ID | Accession | Sequence | SEQ ID NO: |
|---|---|---|---|
| mmu-miR-770-3p | MIMAT0003891 | cgugggccugacguggagcugg | 54 |
| mmu-miR-770-5p | MIMAT0004822 | agcaccacgugucugggccacg | 55 |
| mmu-miR-673-3p | MIMAT0004824 | uccggggcugaguucugugcacc | 56 |
| mmu-miR-673-5p | MIMAT0003739 | cucacagcucugguccuuggag | 57 |
| mmu-miR-493 | MIMAT0004888 | ugaagguccuacugugugccagg | 58 |
| mmu-miR-337-3p | MIMAT0000578 | uucagcuccuauaugaugccu | 59 |
| mmu-miR-337-5p | MIMAT0004644 | gaacggcgucaugcaggaguu | 60 |
| mmu-miR-540-3p | MIMAT0003167 | aggucagaggucgauccugg | 61 |
| mmu-miR-540-5p | MIMAT0004786 | caagggucacccucugacucugu | 62 |
| mmu-miR-665 | MIMAT0003733 | accaggaggcugaggucccu | 63 |
| mmu-miR-431 | MIMAT0001418 | ugucuugcaggccgucaugca | 64 |
| mmu-miR-431* | MIMAT0004753 | caggucgucuugcagggcuucu | 65 |
| mmu-miR-433 | MIMAT0001420 | aucaugaugggcuccucggugu | 66 |
| mmu-miR-433* | MIMAT0001419 | uacggugagccugucauuauuc | 67 |
| mmu-miR-127 | MIMAT0000139 | ucggauccgucugagcuuggcu | 68 |
| mmu-miR-127* | MIMAT0004530 | cugaagcucagagggcucugau | 69 |
| mmu-miR-434-3p | MIMAT0001422 | uuugaaccaucacucgacuccu | 70 |
| mmu-miR-434-5p | MIMAT0001421 | gcucgacucaugguuugaacca | 71 |
| mmu-miR-432 | MIMAT0012771 | ucuuggaguagaucagugggcag | 72 |
| mmu-miR-136 | MIMAT0000148 | acuccauuuguuuugaugaugg | 73 |
| mmu-miR-136* | MIMAT0004532 | aucaucgucucaaaugagucuu | 74 |
| mmu-miR-341 | MIMAT0000588 | ucggucgaucggucggucggu | 75 |
| mmu-miR-1188 | MIMAT0005843 | uggugugagguugggccagga | 76 |
| mmu-miR-370 | MIMAT0001095 | gccugcugggguggaaccuggu | 77 |
| mmu-miR-882 | MIMAT0004847 | aggagagaguuagcgcauuagu | 78 |
| mmu-miR-379 | MIMAT0000743 | ugguagacuauggaacguagg | 79 |
| mmu-miR-411 | MIMAT0004747 | uaguagaccguauagcguacg | 80 |
| mmu-miR-411* | MIMAT0001093 | uauguaacacgguccacuaacc | 81 |
| mmu-miR-299 | MIMAT0004577 | uaugugggacgguaaaccgcuu | 82 |
| mmu-miR-299* | MIMAT0000377 | ugguuuaccgucccacauacau | 83 |
| mmu-miR-380-3p | MIMAT0000745 | uauguaguaugguccacaucuu | 84 |
| mmu-miR-380-5p | MIMAT0000744 | augguugaccauagaacaugcg | 85 |
| mmu-miR-1197 | MIMAT0005858 | uaggacacauggucuacuucu | 86 |
| mmu-miR-323-3p | MIMAT0000551 | cacauuacacggucgaccucu | 87 |
| mmu-miR-323-5p | MIMAT0004638 | aggugguccguggcgcguucgc | 88 |
| mmu-miR-758 | MIMAT0003889 | uuugugaccugguccacua | 89 |
| mmu-miR-329 | MIMAT0000567 | aacacacccagcuaaccuuuuu | 90 |
| mmu-miR-494 | MIMAT0003182 | ugaaacauacacgggaaaccuc | 91 |
| mmu-miR-679 | MIMAT0003455 | ggacugugaggugacucuuggu | 92 |
| mmu-miR-1193 | MIMAT0005851 | uaggucacccguuuuacuauc | 93 |
| mmu-miR-666-3p | MIMAT0004823 | ggcugcagcgugaucgccugcu | 94 |
| mmu-miR-666-5p | MIMAT0003737 | agcgggcacagcugugagagcc | 95 |
| mmu-miR-543 | MIMAT0003168 | aaacauucgcggugcacuucuu | 96 |
| mmu-miR-495 | MIMAT0003456 | aaacaaacauggugcacuucuu | 97 |
| mmu-miR-667 | MIMAT0003734 | ugacaccugccacccagcccaag | 98 |
| mmu-miR-376c | MIMAT0003183 | aacauagaggaaauuucacgu | 99 |
| mmu-miR-376c* | MIMAT0005295 | guggauauuccuucuauguuua | 100 |
| mmu-miR-654-3p | MIMAT0004898 | uaugucugcugaccaucaccuu | 101 |
| mmu-miR-654-5p | MIMAT0004897 | ugguaagcugcagaacaugugu | 102 |
| mmu-miR-376b | MIMAT0001092 | aucauagaggaacauccacuu | 103 |
| mmu-miR-376b* | MIMAT0003388 | guggauauuccuucuaugguua | 104 |
| mmu-miR-376a | MIMAT0000740 | aucguagaggaaaauccacgu | 105 |
| mmu-miR-376a* | MIMAT0003387 | gguagauucuccuucuaugagu | 106 |
| mmu-miR-300 | MIMAT0000378 | uaugcaagggcaagcucucuuc | 107 |
| mmu-miR-300* | MIMAT0004578 | uugaagagagguuauccuuugu | 108 |
| mmu-miR-381 | MIMAT0000746 | uauacaagggcaagcucucugu | 109 |
| mmu-miR-487b | MIMAT0003184 | aaucguacagggucauccacuu | 110 |
| mmu-miR-539 | MIMAT0003169 | ggagaaauuauccuuggugugu | 111 |
| mmu-miR-544 | MIMAT0004941 | auucugcauuuuuagcaagcuc | 112 |
| mmu-miR-382 | MIMAT0000747 | gaaguuguucgugguggauucg | 113 |
| mmu-miR-382* | MIMAT0004691 | ucauucacggacaacacuuuuu | 114 |
| mmu-miR-134 | MIMAT0000146 | ugugacugguugaccagagggg | 115 |
| mmu-miR-668 | MIMAT0003732 | ugucacucggcucggcccacuacc | 116 |
| mmu-miR-485 | MIMAT0003128 | agaggcuggccgugaugaauuc | 117 |
| mmu-miR-485* | MIMAT0003129 | agucauacacggcucuccucuc | 118 |
| mmu-miR-453 | MIMAT0004870 | agguugccucauagugagcuugca | 119 |
| mmu-miR-154 | MIMAT0000164 | uagguuauccguguugccuucg | 120 |
| mmu-miR-154* | MIMAT0004537 | aaucauacacgguugaccuauu | 121 |
| mmu-miR-496 | MIMAT0003738 | ugaguauuacauggccaaucuc | 122 |
| mmu-miR-377 | MIMAT0000741 | aucacacaaaggcaacuuuugu | 123 |
| mmu-miR-541 | MIMAT0003170 | aagggauucugauguuggucacacu | 124 |
| mmu-miR-409-3p | MIMAT0001090 | gaauguugcucggugaaccccu | 125 |
| mmu-miR-409-5p | MIMAT0004746 | agguuacccgagcaacuuugcau | 126 |
| mmu-miR-412 | MIMAT0001094 | uucaccugguccacuagccg | 127 |
| mmu-miR-369-3p | MIMAT0003186 | aauaauacaugguugaucuuu | 128 |
| mmu-miR-369-5p | MIMAT0003185 | agaucgaccguguuauauucgc | 129 |
| mmu-miR-410 | MIMAT0001091 | aauauaacacagauggccugu | 130 |

TABLE 3
Pri-miRNA of human Dlk1-Dio3 region

| | | | SEQ |
|---|---|---|---|
| | | | ID |
| ID | Accession | Sequence | NO: |
| hsa-mir- | MI0005118 | AGGAGCCACCUUCCGAGCCUCCAGUACCA | 131 |
| 770 | | CGUGUCAGGGCCACAUGAGCUGGGCCUCG | |
| | | UGGGCCUGAUGUGGUGCUGGGGCCUCAGG | |
| | | GGUCUGCUCUU | |
| hsa-mir- | MI0003132 | CUGGCCUCCAGGGCUUUGUACAUGGUAGG | 132 |
| 493 | | CUUUCAUUCAUUCGUUUGCACAUUCGGUG | |
| | | AAGGUCUACUGUGUGCCAGGCCCUGUGCC | |
| | | AG | |
| hsa-mir- | MI0000806 | GUAGUCAGUAGUUGGGGGGUGGGAACGGC | 133 |
| 337 | | UUCAUACAGGAGUUGAUGCACAGUUAUCC | |
| | | AGCUCCUAUAUGAUGCCUUUCUUCAUCCC | |
| | | CUUCAA | |
| hsa-mir- | MI0005563 | UCUCCUCGAGGGGUCUCUGCCUCUACCCA | 134 |
| 665 | | GGACUCUUUCAUGACCAGGAGGCUGAGGC | |
| | | CCCUCACAGGCGGC | |
| hsa-mir- | MI0001721 | UCCUGCUUGUCCUGCGAGGUGUCUUGCAG | 135 |
| 431 | | GCCGUCAUGCAGGCCACACUGACGGUAAC | |
| | | GUUGCAGGUCGUCUUGCAGGGCUUCUCGC | |
| | | AAGACGACAUCCUCAUCACCAACGACG | |
| hsa-mir- | MI0001723 | CCGGGGAGAAGUACGGUGAGCCUGUCAUU | 136 |
| 433 | | AUUCAGAGAGGCUAGAUCCUCUGUGUUGA | |
| | | GAAGGAUCAUGAUGGGCUCCUCGGUGUUC | |
| | | UCCAGG | |
| hsa-mir- | MI0000472 | UGUGAUCACUGUCUCCAGCCUGCUGAAGC | 137 |
| 127 | | UCAGAGGGCUCUGAUUCAGAAAGAUCAUC | |
| | | GGAUCCGUCUGAGCUUGGCUGGUCGGAAG | |
| | | UCUCAUCAUC | |
| hsa-mir- | MI0003133 | UGACUCCUCCAGGUCUUGGAGUAGGUCAU | 138 |
| 432 | | UGGGUGGAUCCUCUAUUUCCUUACGUGGG | |
| | | CCACUGGAUGGCUCCUCCAUGUCUUGGAG | |
| | | UAGAUCA | |
| hsa-mir- | MI0000475 | UGAGCCCUCGGAGGACUCCAUUUGUUUUG | 139 |
| 136 | | AUGAUGGAUUCUUAUGCUCCAUCAUCGUC | |
| | | UCAAAUGAGUCUUCAGAGGGUUCU | |
| hsa-mir- | MI0000778 | AGACAGAGAAGCCAGGUCACGUCUCUGCA | 140 |
| 370 | | GUUACACAGCUCACGAGUGCCUGCUGGGG | |
| | | UGGAACCUGGUCUGUCU | |
| hsa-mir- | MI0000787 | AGAGAUGGUAGACUAUGGAACGUAGGCGU | 141 |
| 379 | | UAUGAUUUCUGACCUAUGUAACAUGGUCC | |
| | | ACUAACUCU | |
| hsa-mir- | MI0003675 | UGGUACUUGGAGAGAUAGUAGACCGUAUA | 142 |
| 411 | | GCGUACGCUUUAUCUGUGACGUAUGUAAC | |
| | | ACGGUCCACUAACCCUCAGUAUCAAAUCC | |
| | | AUCCCCGAG | |
| hsa-mir- | MI0000744 | AAGAAAUGGUUUACCGUCCCACAUACAUU | 143 |
| 299 | | UUGAAUAUGUAUGUGGGAUGGUAAACCGC | |
| | | UUCUU | |
| hsa-mir- | MI0000788 | AAGAUGGUUGACCAUAGAACAUGCGCUAU | 144 |
| 380 | | CUCUGUGUCGUAUGUAAUAUGGUCCACAU | |
| | | CUU | |
| hsa-mir- | MI0006656 | ACUUCCUGGUAUUUGAAGAUGCGGUUGAC | 145 |
| 1197 | | CAUGGUGUGUACGCUUUAUUUGUGACGUA | |
| | | GGACACAUGGUCUACUUCUUCUCAAUAUC | |
| | | A | |
| hsa-mir- | MI0000807 | UUGGUACUUGGAGAGAGGUGGUCCGUGGC | 146 |
| 323 | | GCGUUCGCUUUAUUUAUGGCGCACAUUAC | |
| | | ACGGUCGACCUCUUUGCAGUAUCUAAUC | |
| hsa-mir- | MI0003757 | GCCUGGAUACAUGAGAUGGUUGACCAGAG | 147 |
| 758 | | AGCACACGCUUUAUUUGUGCCGUUUGUGA | |
| | | CCUGGUCCACUAACCCUCAGUAUCUAAUG | |
| | | C | |
| hsa-mir- | MI0001725 | GGUACCUGAAGAGAGGUUUUCUGGGUUUC | 148 |
| 329-1 | | UGUUUCUUUAAUGAGGACGAAACACACCU | |
| | | GGUUAACCUCUUUUCCAGUAUC | |
| hsa-mir- | MI0001726 | GUGGUACCUGAAGAGAGGUUUUCUGGGUU | 149 |
| 329-2 | | UCUGUUUCUUUAUUGAGGACGAAACACAC | |
| | | CUGGUUAACCUCUUUUCCAGUAUCAA | |
| hsa-mir- | MI0003134 | GAUACUCGAAGGAGAGGUUGUCCGUGUUG | 150 |
| 494 | | UCUUCUCUUUAUUUAUGAUGAAACAUACA | |
| | | CGGGAAACCUCUUUUUUAGUAUC | |
| hsa-mir- | MI0005565 | UACUUAAUGAGAAGUUGCCCGUGUUUUUU | 151 |
| 543 | | UCGCUUUAUUUGUGACGAAACAUUCGCGG | |
| | | UGCACUUCUUUUUCAGUAUC | |
| hsa-mir- | MI0003135 | UGGUACCUGAAAAGAAGUUGCCCAUGUUA | 152 |
| 495 | | UUUUCGCUUUAUAUGUGACGAAACAAACA | |
| | | UGGUGCACUUCUUUUUCGGUAUCA | |
| hsa-mir- | MI0000776 | AAAAGGUGGAUAUUCCUUCUAUGUUUAUG | 153 |
| 376c | | UUAUUUAUGGUUAAACAUAGAGGAAAUUC | |
| | | CACGUUUU | |
| hsa-mir- | MI0003529 | GGUAUUUAAAAGGUAGAUUUUCCUUCUAU | 154 |
| 376a-2 | | GGUUACGUGUUUGAUGGUUAAUCAUAGAG | |
| | | GAAAAUCCACGUUUUCAGUAUC | |
| hsa-mir- | MI0003676 | GGGUAAGUGGAAAGAUGGUGGGCCGCAGA | 155 |
| 654 | | ACAUGUGCUGAGUUCGUGCCAUAUGUCUG | |
| | | CUGACCAUCACCUUUAGAAGCCC | |
| hsa-mir- | MI0002466 | CAGUCCUUCUUUGGUAUUUAAAACGUGGA | 156 |
| 376b | | UAUUCCUUCUAUGUUUACGUGAUUCCUGG | |
| | | UUAAUCAUAGAGGAAAAUCCAUGUUUUCA | |
| | | GUAUCAAAUGCUG | |
| hsa-mir- | MI0000784 | UAAAAGGUAGAUUCUCCUUCUAUGAGUAC | 157 |
| 376a-1 | | AUUAUUUAUGAUUAAUCAUAGAGGAAAAU | |
| | | CCACGUUUUC | |
| hsa-mir- | MI0005525 | UGCUACUUGAAGAGAGGUAAUCCUUCACG | 158 |
| 300 | | CAUUUGCUUUACUUGCAAUGAUUAUACAA | |
| | | GGGCAGACUCUCUCUGGGGAGCAAA | |
| hsa-mir- | MI0003844 | UUUGGUACUUGAAGAGAGGAUACCCUUUG | 159 |
| 1185-1 | | UAUGUUCACUUGAUUAAUGGCGAAUAUAC | |
| | | AGGGGGAGACUCUUAUUUGCGUAUCAAA | |
| hsa-mir- | MI0003821 | UUUGGUACUUAAAGAGAGGAUACCCUUUG | 160 |
| 1185-2 | | UAUGUUCACUUGAUUAAUGGCGAAUAUAC | |
| | | AGGGGGAGACUCUCAUUUGCGUAUCAAA | |
| hsa-mir- | MI0000789 | UACUUAAAGCGAGGUUGCCCUUUGUAUAU | 161 |
| 381 | | UCGGUUUAUUGACAUGGAAUAUACAAGGG | |
| | | CAAGCUCUCUGUGAGUA | |
| hsa-mir- | MI0003530 | UUGGUACUUGGAGAGUGGUUAUCCCUGUC | 162 |
| 487b | | CUGUUCGUUUUGCUCAUGUCGAAUCGUAC | |
| | | AGGGUCAUCCACUUUUUCAGUAUCAA | |
| hsa-mir- | MI0003514 | AUACUUGAGGAGAAAUUAUCCUUGGUGUG | 163 |
| 539 | | UUCGCUUUAUUUAUGAUGAAUCAUACAAG | |
| | | GACAAUUUCUUUUUGAGUAU | |
| hsa-mir- | MI0005540 | GUGCUUAAAGAAUGGCUGUCCGUAGUAUG | 164 |
| 889 | | GUCUCUAUAUUUAUGAUGAUUAAUAUCGG | |
| | | ACAACCAUUGUUUUAGUAUCC | |
| hsa-mir- | MI0003515 | AUUUUCAUCACCUAGGGAUCUUGUUAAAA | 165 |
| 544 | | AGCAGAUUCUGAUUCAGGGACCAAGAUUC | |
| | | UGCAUUUUUAGCAAGUUCUCAAGUGAUGC | |
| | | UAAU | |
| hsa-mir- | MI0003677 | AACUAUGCAAGGAUAUUUGAGGAGAGGUU | 166 |
| 655 | | AUCCGUGUUAUGUUCGCUUCAUUCAUCAU | |
| | | GAAUAAUACAUGGUUAACCUCUUUUUGAA | |
| | | UAUCAGACUC | |
| hsa-mir- | MI0002471 | GGUACUUGAAGAGUGGUUAUCCCUGCUGU | 167 |
| 487a | | GUUCGCUUAAUUUAUGACGAAUCAUACAG | |
| | | GGACAUCCAGUUUUUCAGUAUC | |
| hsa-mir- | MI0000790 | UACUUGAAGAGAAGUUGUUCGUGGUGGAU | 168 |
| 382 | | UCGCUUUACUUAUGACGAAUCAUUCACGG | |
| | | ACAACACUUUUUUCAGUA | |
| hsa-mir- | MI0000474 | CAGGGUGUGUGACUGGUUGACCAGAGGGG | 169 |
| 134 | | CAUGCACUGUGUUCACCCUGUGGGCCACC | |
| | | UAGUCACCAACCCUC | |
| hsa-mir- | MI0003761 | GGUAAGUGCGCCUCGGGUGAGCAUGCACU | 170 |
| 668 | | UAAUGUGGGUGUAUGUCACUCGGCUCGGC | |
| | | CCACUACC | |
| hsa-mir- | MI0002469 | ACUUGGAGAGAGGCUGGCCGUGAUGAAUU | 171 |
| 485 | | CGAUUCAUCAAAGCGAGUCAUACACGGCU | |
| | | CUCCUCUCUUUUAGU | |
| hsa-mir- | MI0001727 | GCAGGAAUGCUGCGAGCAGUGCCACCUCA | 172 |
| 453 | | UGGUACUCGGAGGGAGGUUGUCCGUGGUG | |
| | | AGUUCGCAUUAUUUAAUGAUGC | |
| hsa-mir- | MI0000480 | GUGGUACUUGAAGAUAGGUUAUCCGUGUU | 173 |
| 154 | | GCCUUCGCUUUAUUUGUGACGAAUCAUAC | |
| | | ACGGUUGACCUAUUUUUCAGUACCAA | |
| hsa-mir- | MI0003136 | CCCAAGUCAGGUACUCGAAUGGAGGUUGU | 174 |
| 496 | | CCAUGGUGUGUUCAUUUUAUUUAUGAUGA | |
| | | GUAUUACAUGGCCAAUCUCCUUUCGGUAC | |
| | | UCAAUUCUUCUUGGG | |
| hsa-mir- | MI0000785 | UUGAGCAGAGGUUGCCCUUGGUGAAUUCG | 175 |
| 377 | | CUUUAUUUAUGUUGAAUCACACAAAGGCA | |
| | | ACUUUUGUUUG | |
| hsa-mir- | MI0005539 | ACGUCAGGGAAAGGAUUCUGCUGUCGGUC | 176 |
| 541 | | CCACUCCAAAGUUCACAGAAUGGGUGGUG | |
| | | GGCACAGAAUCUGGACUCUGCUUGUG | |
| hsa-mir- | MI0001735 | UGGUACUCGGGGAGAGGUUACCCGAGCAA | 177 |
| 409 | | CUUUGCAUCUGGACGACGAAUGUUGCUCG | |
| | | GUGAACCCCUUUUCGGUAUCA | |
| hsa-mir- | MI0002464 | CUGGGGUACGGGGAUGGAUGGUCGACCAG | 178 |
| 412 | | UUGGAAAGUAAUUGUUUCUAAUGUACUUC | |
| | | ACCUGGUCCACUAGCCGUCCGUAUCCGCU | |
| | | GCAG | |
| hsa-mir- | MI0000777 | UUGAAGGGAGAUCGACCGUGUUAUAUUCG | 179 |
| 369 | | CUUUAUUGACUUCGAAUAAUACAUGGUUG | |
| | | AUCUUUUCUCAG | |
| hsa-mir- | MI0002465 | GGUACCUGAGAAGAGGUUGUCUGUGAUGA | 180 |
| 410 | | GUUCGCUUUUAUUAAUGACGAAUAUAACA | |
| | | CAGAUGGCCUGUUUUCAGUACC | |
| hsa-mir- | MI0003678 | CUGAAAUAGGUUGCCUGUGAGGUGUUCAC | 181 |
| 656 | | UUUCUAUAUGAUGAAUAUUAUACAGUCAA | |
| | | CCUCUUUCCGAUAUCGAAUC | |

TABLE 4
Mature-miRNA of human Dlk1-Dio3 region

| ID | Accession | Sequence | SEQ ID NO: |
|---|---|---|---|
| hsa-miR-770-5p | MIMAT0003948 | uccaguaccacgugucagggcca | 182 |
| hsa-miR-493 | MIMAT0003161 | ugaaggucuacugugugccagg | 183 |
| hsa-miR-493* | MIMAT0002813 | uuguacaugguaggcuuucauu | 184 |
| hsa-miR-337-5p | MIMAT0004695 | gaacggcuucauacaggaguu | 185 |
| hsa-miR-337-3p | MIMAT0000754 | cuccuauaugaugccuuucuuc | 186 |
| hsa-miR-665 | MIMAT0004952 | accaggaggcugaggccccu | 187 |
| hsa-miR-431 | MIMAT0001625 | ugucuugcaggccgucaugca | 188 |
| hsa-miR-431* | MIMAT0004757 | caggucgucuugcagggcuucu | 189 |
| hsa-miR-433 | MIMAT0001627 | aucaugaugggcuccucggugu | 190 |
| hsa-miR-127-5p | MIMAT0004604 | cugaagcucagagggcucugau | 191 |
| hsa-miR-127-3p | MIMAT0000446 | ucggauccgucugagcuuggcu | 192 |
| hsa-miR-432 | MIMAT0002814 | ucuuggaguaggucauugggugg | 193 |
| hsa-miR-432* | MIMAT0002815 | cuggauggcuccuccaugucu | 194 |
| hsa-miR-136 | MIMAT0000448 | acuccauuuguuuugaugaugga | 195 |
| hsa-miR-136* | MIMAT0004606 | caucaucgucucaaaugagucu | 196 |
| hsa-miR-370 | MIMAT0000722 | gccugcugggguggaaccuggu | 197 |
| hsa-miR-379 | MIMAT0000733 | ugguagacuauggaacguagg | 198 |
| hsa-miR-379* | MIMAT0004690 | uauguaacaugguccacuaacu | 199 |
| hsa-miR-411 | MIMAT0003329 | uaguagaccguauagcguacg | 200 |
| hsa-miR-411* | MIMAT0004813 | uauguaacacgguccacuaacc | 201 |
| hsa-miR-299-5p | MIMAT0002890 | ugguuuaccgucccacauacau | 202 |
| hsa-miR-299-3p | MIMAT0000687 | uaugugggaugguaaaccgcuu | 203 |
| hsa-miR-380 | MIMAT0000735 | uauguaauaugguccacaucuu | 204 |
| hsa-miR-380* | MIMAT0000734 | ugguugaccauagaacaugcgc | 205 |
| hsa-miR-1197 | MIMAT0005955 | uaggacacauggucuacuucu | 206 |
| hsa-miR-323-5p | MIMAT0004696 | aggugguccguggcgcguucgc | 207 |
| hsa-miR-323-3p | MIMAT0000755 | cacauuacacggucgaccucu | 208 |
| hsa-miR-758 | MIMAT0003879 | uuugugaccugguccacuaacc | 209 |
| hsa-miR-329 | MIMAT0001629 | aacacaccugguuaaccucuuu | 210 |
| hsa-miR-494 | MIMAT0002816 | ugaaacauacacgggaaaccuc | 211 |
| hsa-miR-543 | MIMAT0004954 | aaacauucgcggugcacuucuu | 212 |
| hsa-miR-495 | MIMAT0002817 | aaacaaacauggugcacuucuu | 213 |
| hsa-miR-376c | MIMAT0000720 | aacauagaggaaauuccacgu | 214 |
| hsa-miR-376a | MIMAT0000729 | aucauagaggaaaauccacgu | 215 |
| hsa-miR-654-5p | MIMAT0003330 | uggugggccgcagaacaugugc | 216 |
| hsa-miR-654-3p | MIMAT0004814 | uaugucugcugaccaucaccuu | 217 |
| hsa-miR-376b | MIMAT0002172 | aucauagaggaaaauccauguu | 218 |
| hsa-miR-376a | MIMAT0000729 | aucauagaggaaaauccacgu | 219 |
| hsa-miR-376a* | MIMAT0003386 | guagauucuccuucuaugagua | 220 |
| hsa-miR-300 | MIMAT0004903 | uauacaagggcagacucucucu | 221 |
| hsa-miR-1185 | MIMAT0005798 | agaggauacccuuuguauguu | 222 |
| hsa-miR-381 | MIMAT0000736 | uauacaagggcaagcucucugu | 223 |
| hsa-miR-487b | MIMAT0003180 | aaucguacagggucauccacuu | 224 |
| hsa-miR-539 | MIMAT0003163 | ggagaaauuauccuuggugugu | 225 |
| hsa-miR-889 | MIMAT0004921 | uuaauaucggacaaccauugu | 226 |
| hsa-miR-544 | MIMAT0003164 | auucugcauuuuuagcaaguuc | 227 |
| hsa-miR-655 | MIMAT0003331 | auaauacaugguuaaccucuuu | 228 |
| hsa-miR-487a | MIMAT0002178 | aaucauacagggacauccaguu | 229 |
| hsa-miR-382 | MIMAT0000737 | gaaguuguucgugguggauucg | 230 |
| hsa-miR-134 | MIMAT0000447 | ugugacugguugaccagagggg | 231 |
| hsa-miR-668 | MIMAT0003881 | ugucacucggcucggcccacuac | 232 |
| hsa-miR-485-5p | MIMAT0002175 | agaggcuggccgugaugaauuc | 233 |
| hsa-miR-485-3p | MIMAT0002176 | gucauacacggcucuccucucu | 234 |
| hsa-miR-453 | MIMAT0001630 | agguuguccguggugaguucgca | 235 |
| hsa-miR-154 | MIMAT0000452 | uagguuauccguguugccuucg | 236 |
| hsa-miR-154* | MIMAT0000453 | aaucauacacgguugaccuauu | 237 |
| hsa-miR-496 | MIMAT0002818 | ugaguauuacauggccaaucuc | 238 |
| hsa-miR-377 | MIMAT0000730 | aucacacaaaggcaacuuuugu | 239 |
| hsa-miR-377* | MIMAT0004689 | agagguugcccuuggugaauuc | 240 |
| hsa-miR-541 | MIMAT0004920 | uggugggcacagaaucuggacu | 241 |
| hsa-miR-541* | MIMAT0004919 | aaaggauucugcugucggucccacu | 242 |
| hsa-miR-409-5p | MIMAT0001638 | agguuacccgagcaacuuugcau | 243 |
| hsa-miR-409-3p | MIMAT0001639 | gaauguugcucggugaaccccu | 244 |
| hsa-miR-412 | MIMAT0002170 | acuucaccugguccacuagccgu | 245 |
| hsa-miR-369-5p | MIMAT0001621 | agaucgaccguguuauauucgc | 246 |
| hsa-miR-369-3p | MIMAT0000721 | aauaauacaugguugaucuuu | 247 |
| hsa-miR-410 | MIMAT0002171 | aauauaacacagauggccugu | 248 |
| hsa-miR-656 | MIMAT0003332 | aauauuauacagucaaccucu | 249 |

In the present invention, genes located in imprinted region are preferably genes located in the Dlk1-Dio3 region. Examples of such genes include Dlk1, Gtl2/Meg3, Rtl1, Rtl1as, Meg8/Rian, Meg9/Mirg, and Dio3. More preferable examples of the genes are imprinting genes that are expressed from only a maternally derived chromosome, which are shown in Table 5.

Examples of a method for detecting the expression of the above genes include, but are not particularly limited to, Northern blotting, Southern blotting, hybridization such as Northern hybridization, Southern hybridization, and in situ hybridization, RNase protection assay, a PCR method, quantitative PCR, a real-time PCR method, and a microarray method.

Detection can be performed by microarray method containing following steps of (i) extracting total RNA containing mRNA from a biological sample, (ii) obtaining mRNA using a poly T column, (iii) synthesizing cDNA by a reverse transcription reaction, (iv) amplifying using a phage or a PCR cloning method, and then (v) performing hybridization with a probe consisting of about 20 mer-70 mer or a larger size complementary to the target DNA or by quantitative PCR using about 20 mer-30 mer primers, for example. As a label for hybridization or PCR, a fluorescent label can be used. As such a fluorescent label, cyan, fluorescamine, rhodamine, or a derivative thereof such as Cy3, Cy5, FITC, and TRITC can be used.

The number of a gene to be detected may be any number and is at least 1, at least 2, or at least 3. More preferably the number of such gene is 4.

TABLE 5
Maternally-derived genomic imprinting
genes of Dlk1-Dio3 region

Accession NO

| Gene name | Mouse | Human |
|---|---|---|
| Gtl2/MEG3 | NR_003633 | NR_002766 |
| | (SEQ ID No: 270) | (SEQ ID NO: 274) |
| Rtl1as/anti-Peg11 | NR_002848 | — |
| | (SEQ ID NO: 271) | |
| Rian/MEG8 | NR_028261 | NR_024149 |
| | (SEQ ID NO: 272) | (SEQ ID NO: 275) |
| Mirg/Meg9 | NR_028265 | — |
| | (SEQ ID NO: 273) | |

Upon screening iPS cells having unlimitedly high differentiation potency and being capable of germline transmission, a value detected by the above method for control cells which are iPS cells or embryonic stem cells (ES cells) known to perform germline transmission is designated as the reference value (positive reference value). Subject iPS cells for which the value is equivalent to or higher than the positive reference value may be selected as iPS cells capable of germline transmission.

Similarly, a value detected by the above method for control cells which are iPS cells or embryonic stem cells (ES cells) that are known not to perform germline transmission is designated as the reference gene (negative reference gene). Subject iPS cells for which the value is higher than the negative reference value may be selected as iPS cells capable of germline transmission.

Another embodiment involves preparing Table 6 in advance using a series of cells known to perform or known not to be able to perform germline transmission and then designating the reference value so that the values for each or both sensitivity and specificity shown in Table 6 are 0.9 or more, preferably 0.95 or more, and more preferably 0.99 or more. When a value detected for subject iPS cells by the above method is equivalent to or higher than the reference value, the subject iPS cells can be screened for as iPS cells capable of germline transmission. Particularly preferably, the values for both sensitivity and specificity are 1. Here, a result in which both sensitivity and specificity are 1 indicates that the reference value is an identical reference value that will have neither a false-positive result nor a false-negative result.

TABLE 6

| | Number of | Number of |
|---|---|---|
| | iPS cells | iPS cells |
| | capable of | incapable of |
| | germline | germline |
| | transmission | transmission |
| Number of cell lines for | A | C |
| which the detected value was | | |
| the same as or higher than the | | |
| reference gene | | |
| Number of cell lines for | B | D |
| which the detected value was | | |
| lower than the reference gene | | |
| | Sensitivity = | Specificity = |
| | A/(A + B) | D/(C + D) |

Furthermore, in the present invention, method for screening iPS cells capable of germline transmission may also be performed by detecting methylation of DNA in region controlling expression of the gene located in the Dlk1-Dio3 region. At this time, an example of a region to be detected is a region that is referred to as a CpG island, which is the region having a high content of sequence consisting of cytosine and guanine, located between the region encoding Dlk1 and the region encoding Gtl2/MEG3, wherein its DNA methylation state in a maternally derived chromosome is different from that in a paternally derived chromosome. A preferable example of such region is an intergenic differentially methylated region (IG-DMR) or MEG3-DMR (Gtl2-DMR). Examples of the above IG-DMR and MEG3-DMR include, but are not particularly limited to, regions as described in Cytogenet Genome Res 113:223-229, (2006), Nat Genet. 40:237-42, (2008) or Nat Genet. 35:97-102. (2003). A more specific example of the above IG-DMR is, in the case of mice, a region with a length of 351 bp ranging from nucleotide 80479 to nucleotide 80829 in the AJ320506 sequence of NCBI.

Examples of a method for detecting DNA methylation include methods that involve cleaving a subject recognition sequence using a restriction enzyme and methods that involve hydrolyzing unmethylated cytosine using bisulfite.

The former methods use a methylation-sensitive or -insensitive restriction enzyme, which is based on the fact that if a nucleotide in a recognition sequence is methylated, the cleaving activity of the restriction enzyme is altered. The thus generated DNA fragment is subjected to electrophoresis and then the fragment length of interest is measured by Southern blotting or the like, so that a methylated site is detected. On the other hand, the latter methods include a method that involves performing bisulfite treatment, PCR, and then sequencing, a method that involves using methylation-specific oligonucleotide (MSO) microarrays, or methylation-specific PCR that involves causing PCR primers to recognize a difference between a sequence before bisulfite treatment and the sequence after bisulfite treatment and then determining the presence or the absence of methylated DNA based on the presence or the absence of PCR products. In addition to these methods, by chromosome immunoprecipitation using a DNA methylation-specific antibody, DNA-methylated regions can be detected from specific regions by extracting DNA sequences within DNA-methylated regions, performing PCR, and then performing sequencing.

Upon screening iPS cells having unlimitedly high differentiation potency and being capable of germline transmission, subject iPS cells in which the subject region in one chromosome is in a DNA-methylated state, but the same region in homologous chromosome is not in a DNA-methylated state as detected by the above method can be selected as iPS cells having unlimitedly high differentiation potency or capable of germline transmission. Here, the expression, “the subject region in one chromosome is in a DNA-methylated state, but the same region in homologous chromosome is not in a DNA-methylated state” refers to, for example, a state in which the detected methylated CpGs in the subject region account for 30% or more and 70% or less, preferably 40% or more and 60% or less, more preferably 45% or more and 55% or less, and particularly preferably 50% of all detected CpGs. In a more preferable embodiment, a paternally derived chromosome alone is methylated and the same region of the maternally derived chromosome in the same cell is not methylated. As a result, it is desirable to select iPS cells for which detected methylated CpGs account for 50% of all detected CpGs.

As an example of a method for detecting the percentage of methylated CpGs, in the case of using a restriction enzyme recognizing unmethylated DNA, the percentage accounted for methylated DNAs can be calculated by comparing the amount of unfragmented DNA with fragmented DNA determined by Southern blotting. Meanwhile, in the case of the bisulfite method, arbitrarily selected chromosomes are sequenced. Hence, the percentage can be calculated by repeatedly sequencing a template to which a PCR product has been cloned a plurality of times such as 2 or more times, preferably 5 or more times, and more preferably 10 or more times and then comparing the number of sequenced clones with the number of clones for which DNA methylation has been detected. When a pyro-sequencing method is employed, the percentage can also be directly determined by measuring amount of cytosine or thymine (the amount of cytosine means amount of methylated DNAs and the amount of thymine means amount of unmethylated DNAs). Also, in the case of a chromosome immunoprecipitation method using a DNA methylation-specific antibody, the amount of precipitated DNA of interest and the amount of DNA before precipitation are detected by PCR and then compared, so that the percentage accounted for by methylated DNAs can be detected.

### Kit for Screening of iPS Cells

The kit for screening iPS cells according to the present invention contains a reagent for miRNA measurement, a reagent for gene measurement, or a reagent for measuring DNA methylation for the above detection method.

Examples of the reagent for miRNA measurement are probe or primer nucleic acids, including the whole or partial sequences of the RNA listed in Tables 1, 2, 3, and 4 or cDNA encoding the RNA. The size of the probe is generally at least 15 nucleotides, preferably at least 20 nucleotides, for example 20-30 nucleotides, 30-70 nucleotides, 70-100 nucleotide or more, etc.

The reagent for miRNA measurement also may contain, as an alternative to RNA having a sequence complementary to the nucleotide sequence of an miRNA shown in any of Tables 1-4 above, an artificial nucleic acid such as LNA (locked nucleic acid; also LNA referred to as bridged nucleic acid (BNA)) or PNA (peptide nucleic acid) as a probe.

A reagent for gene measurement can contain nucleic acid probes of a size of about 20 mer-70 mer or more in size that are fragments of target DNA or mRNA of an imprinting gene described in Table 5 above or nucleic acids complementary to the fragments, or a primer set or primers of about 20 mer-30 mer in size derived from said fragments and nucleic acids complementary thereto.

The kit can also contain microarrays prepared by binding the above-described probes to carriers, such as glassor polymers.

A reagent for DNA methylation measurement contains a reagent and microarrays to be used for an MSO (methylation-specific oligonucleotide) microarray method for detection of methylation of cytosine nucleotides using a bisulfite reaction (Izuho Hatada, Experimental Medicine, Vol. 24, No. 8 (Extra Number), pp. 212-219 (2006), YODOSHA (Japan)). In the bisulfite method, a single-stranded DNA is treated with bisulfite (sodium sulfite), so as to convert cytosine to uracil, but methylated cytosine is not converted to uracil. In a methylation specific oligonucleotide (MSO) microarray method, methylation is detected using a bisulfite reaction. In this method, PCR is performed for DNA treated with bisulfite by selecting sequences (containing no CpG sequences) that remain unaltered regardless of methylation as primers. As a result, unmethylated cytosine is amplified as thymine and methylated cytosine is amplified as cytosine. Oligonucleotides complementary to sequences in which thymine has been altered from unmethylated cytosine (in the case of unmethylated cytosine) and oligonucleotides complementary to sequences in which cytosine has remained unaltered (in the case of methylated cytosine) are immobilized to carriers of microarrays. The thus amplified DNA is fluorescence-labeled and then hybridized to the microarrays. Methylation can be quantitatively determined based on the occurrence of hybridization. A kit for determining a DNA methylation state of IG-DMR and/or Gtl2/MEG3-DMR for screening of induced pluripotent stem cells can contain a methylation-sensitive restriction enzyme, or a bisulfite reagent, and nucleic acids for amplification of IG-DMR and/or Gtl2/MEG3-DMR.

Example of the methylation-sensitive restriction enzymes include, but are not limited to, AatII, AccII, BssHII, ClaI, CpoI, Eco52I, HaeII, MluI, NaeI, NotI, NsbI, PvuI, SacII, SalI, etc.

The kit for screening iPS cells of the present invention can also contain a reagent for miRNA extraction, a reagent for gene extraction, or a reagent for chromosome extraction, for example. Also, a kit for diagnosis of the present invention may contain means for discrimination analysis such as documents or instructions containing procedures for discrimination analysis, a program for implementing the procedures for discrimination analysis by a computer, the program list, a recording medium containing the program recorded therein, which is readable by the computer (e.g., flexible disk, optical disk, CD-ROM, CD-R, and CD-RW), and an apparatus or a system (e.g., computer) for implementation of discrimination analysis.

The present invention will be further described in detail by examples as follows, but the scope of the present invention is not limited by these examples.

### EXAMPLES

### Mouse ES and iPS Cells

Mouse ES cells (RF8, Nanog ES, and Fbx(−/−)ES) shown in Table 7 were cultured and sample iPS cells were established and cultured by conventional methods (Takahashi K and Yamanaka S, Cell 126 (4), 663, 2006; Okita K, et al., Nature 448 (7151), 313, 2007; Nakagawa M, et al., Nat Biotechnol 26 (1), 101, 2008, Aoi, T. et al., Science 321, 699-702, 2008; and Okita K, et al., Science 322, 949, 2008). Also, Table 7 shows the results of studying the generation of chimeric mice from each cell and the presence or the absence of germline transmission according to conventional methods. Here, “origin” indicates somatic cells serving as origins, “MEF” indicates Mouse Embryonic Fibroblast, “TTF” indicates Tail-Tip Fibroblast, “Hep” indicates hepatocytes, and “Stomach” indicates gastric epithelial cells. Also regarding “Transgene,” “O” indicates Oct3/4, “S” indicates Sox2, “M” indicates c-Myc, and “K” indicates Klf4. Furthermore, “no (plasmid OSMK)” indicates that iPS cells were prepared by a plasmid method and no transgene was incorporated into a chromosome.

TABLE 7
List of cells

| | Cell | | | Adult | |
|---|---|---|---|---|---|
| Clone name | type | Origin | Transgene | chimera | Germline |
| RF8 | ES | blastocyst | — | (Yes) | (Yes) |
| Nanog ES | | | — | (Yes) | (Yes) |
| Fbx(−/−)ES | | | — | (Yes) | (Yes) |
| 20D17 | iPS | MEF | OSMK | Yes | Yes |
| 38C2 | | | OSMK | Yes | No |
| 38D2 | | | OSMK | Yes | No |
| 178B2 | | | OSK | Yes | No |
| 178B5 | | | OSK | Yes | Yes |
| 212C5 | | TTF | OSMK | Yes | No |
| 212C6 | | | OSMK | Yes | No |
| 335D1 | | | OSK | Yes | No |
| 335D3 | | | OSK | Yes | No |
| 256H13 | | | OSK | Yes | No |
| 256H18 | | | OSK | Yes | No |
| 98A1 | | Hep | OSMK | Yes | No |
| 103C1 | | | OSMK | Yes | Yes |
| 99-1 | | Stomach | OSMK | Yes | Yes |
| 99-3 | | | OSMK | Yes | Yes |
| 492B4 | | MEF | no (plasmid | Yes | Yes |
| | | | OSMK) | | |
| 492B9 | | | no (plasmid | Yes | No |
| | | | OSMK) | | |
| Fbx iPS 10-6 | | MEF | 10 factors | No | N.D. |
| Fbx iPS 4-7 | | | OSMK | No | N.D. |
| Fbx iPS 4-3 | | TTF | OSMK | No | N.D. |
| Fbx iPS WT1 | | | OSMK | No | N.D. |
| SNL feeder | Soma | | | | |
| MEF | | | | | |
| TTF | | | | | |
| Hepatocyte | | | | | |
| Stomach | | | | | |

### Human ES and iPS Cells

Human ES cells (KhES1, KhES3, H1 and H9) were cultured, and iPS cell samples were established and cultured by conventional methods (Suemori H, et al., Biochem Biophys Res Commun, 345, 926-32, 2006, Thomson J A, et al., 282, 1145-7, 1998, US2009/0047263 and WO2010/013359). These cells were listed in Table 8, wherein “HDF” indicates Human Embryonic Fibroblast.

TABLE 8
List of cells

| Clone name | Cell type | Origin | Transgene |
|---|---|---|---|
| KhES1 | ES | blastocyst | — |
| KhES3 | | | — |
| H1 | | | — |
| H9 | | | — |
| 201B2 | iPS | HDF | OSMK |
| 201B6 | | | OSMK |
| 201B7 | | | OSMK |
| 253G1 | | | OSK |
| 253G4 | | | OSK |
| TIG103-4F4 | | | OSMK |
| TIG107-4F1 | | | OSK |
| TIG107-3F1 | | | OSMK |
| TIG108-4F3 | | | OSMK |
| TIG109-4F1 | | | OSMK |
| TIG114-4F1 | | | OSMK |
| TIG118-4F1 | | | OSMK |
| TIG120-4F1 | | | OSMK |
| TIG121-4F4 | | | OSMK |
| 1375-4F1 | | | OSMK |
| 1377-4F1 | | | OSMK |
| 1392-4F2 | | | OSMK |
| 1488-4F1 | | | OSMK |
| 1503-4F1 | | | OSMK |
| 1687-4F2 | | | OSMK |
| DP31-4F1 | | dental pulp | OSMK |
| 225C7 | | fetal HDF | OSMK |
| 246G1 | | BJ cell | OSMK |

Confirmation of microRNA Expression in Mouse Cells

Profiling of the expression of microRNA expressed in mouse cells shown in Table 7 was performed using microRNA microarrays (Agilent).

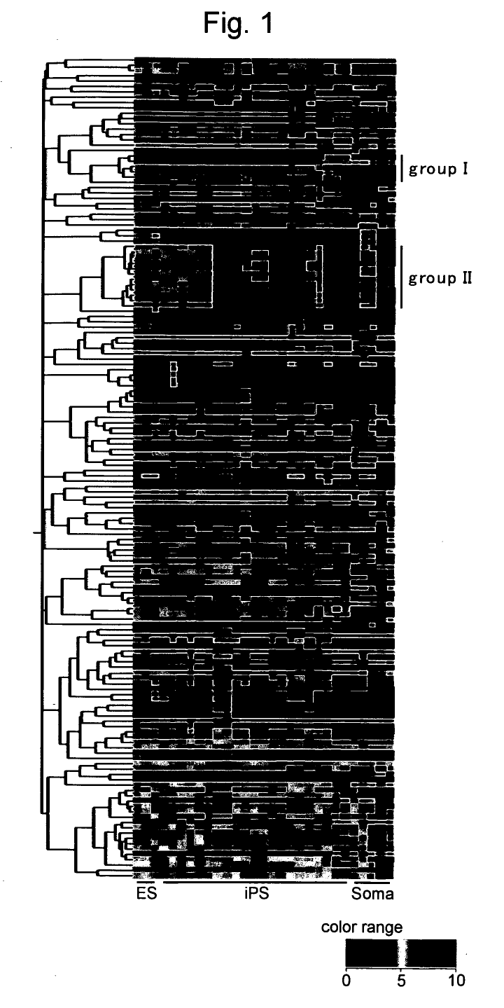

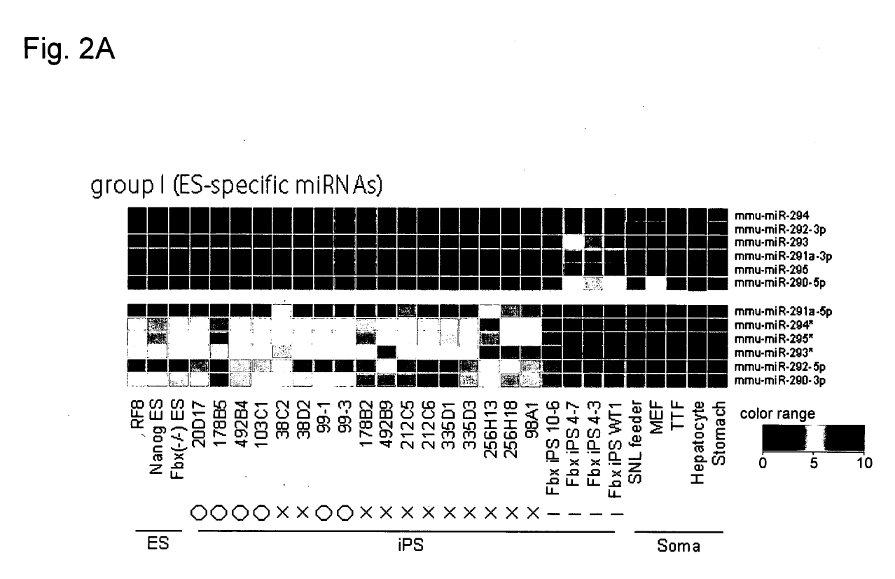

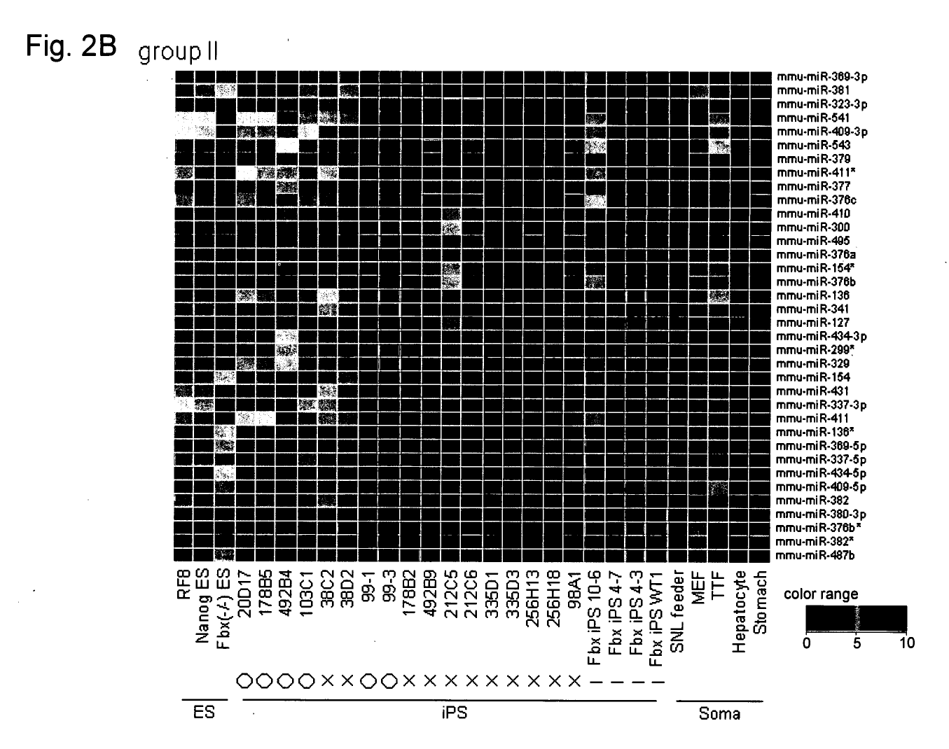

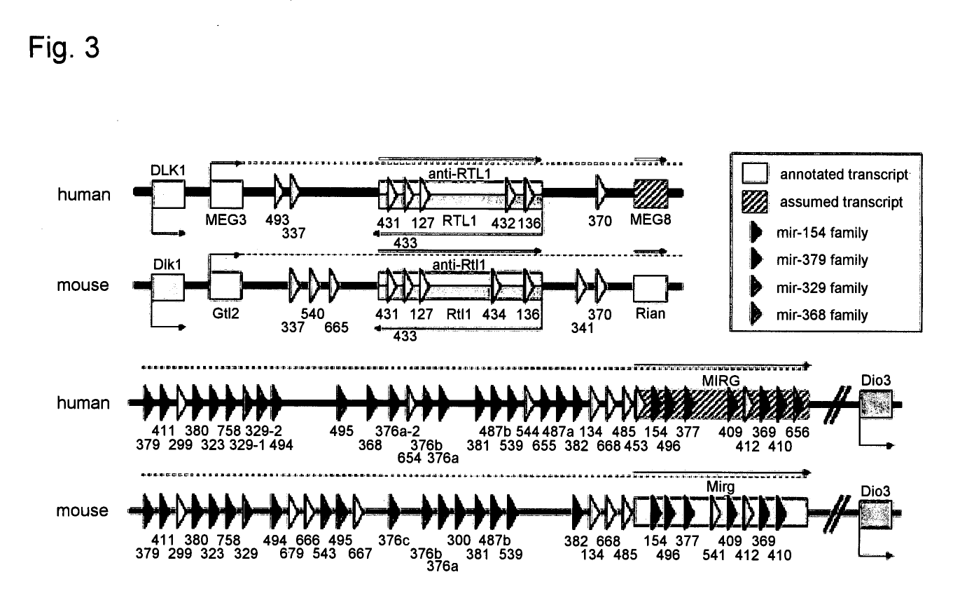

211 probes determined to be ineffective for all the 29 samples were removed from 672 miRNA array probes. Hierarchical clustering was performed for a total of 461 probes. The results are shown in FIG. 1. Group I miRNA not expressed in somatic cells but expressed in ES cells and iPS cells and Group II miRNA expressed in various manners among iPS cells were extracted. Group I is shown in FIG. 2A and Table 9 and Group II is shown in FIG. 2B and Table 10. When Group II miRNA was analyzed, all members were found to be contained in the miRNA cluster of chromosome 12.

Group I miRNA was expressed to an extent equivalent to that in the case of ES cells in the case of iPS cell clones contributing to the birth of chimeric mice, but in the case of 4 clones of Fbx iPS cells not contributing to the birth of chimeric mice, only low expression levels were detected, compared with the case of ES cells. Thus, it was suggested that Group I miRNA can be used as a marker for iPS cells contributing to the birth of chimeric mice.

Group II miRNA was expressed in all clones (20D17, 178B5, 492B4, and 103C1) for which germline transmission could be confirmed, excluding 2 clones (99-1 and 99-3) of gastric-epithelial-cell-derived iPS cells. Also, among iPS clones prepared from MEF, the expression of Group II miRNA was observed in 2 clones (38C2 and 38D2) for which no germline transmission could be confirmed, but Group II miRNA was never expressed or expressed at levels lower than that in the case of ES cells in iPS clones prepared from TTF. It was suggested by the results that examination of Group II miRNA as a marker for iPS cells that are very similar to ES cells in which germline transmission occurs is useful.

TABLE 9
Group I mouse miRNA

| | | | SEQ |
|---|---|---|---|
| | | | ID |
| ID | Accession | Sequence | NO: |
| mmu-miR-290-5p | MIMAT0000366 | acucaaacuaugggggcac | 250 |
| | | uuu | |
| mmu-miR-290-3p | MIMAT0004572 | aaagugccgccuaguuuua | 251 |
| | | agccc | |
| mmu-miR-291a- | MIMAT0000367 | caucaaaguggaggcccuc | 252 |
| 5p | | ucu | |
| mmu-miR-291a- | MIMAT0000368 | aaagugcuuccacuuugug | 253 |
| 3p | | ugc | |
| mmu-miR-292-5p | MIMAT0000369 | acucaaacugggggcucuu | 254 |
| | | uug | |
| mmu-miR-292-3p | MIMAT0000370 | aaagugccgccagguuuug | 255 |
| | | agugu | |
| mmu-miR-293 | MIMAT0000371 | agugccgcagaguuuguag | 256 |
| | | ugu | |
| mmu-miR-293* | MIMAT0004573 | acucaaacugugugacauu | 257 |
| | | uug | |
| mmu-miR-294 | MIMAT0000372 | aaagugcuucccuuuugug | 258 |
| | | ugu | |
| mmu-miR-294* | MIMAT0004574 | acucaaaauggaggcccua | 259 |
| | | ucu | |
| mmu-miR-295 | MIMAT0000373 | aaagugcuacuacuuuuga | 260 |
| | | gucu | |
| mmu-miR-295* | MIMAT0004575 | acucaaauguggggcacac | 261 |
| | | uuc | |

TABLE 10
Group II mouse miRNA

| ID | Accession | Sequence | SEQ ID NO: |
|---|---|---|---|
| mmu-miR-337-3p | MIMAT0004644 | gaacggcgucaugcaggaguu | 59 |
| mmu-miR-337-5p | MIMAT0000578 | uucagcuccuauaugaugccu | 60 |
| mmu-miR-431 | MIMAT0001418 | ugucuugcaggccgucaugca | 64 |
| mmu-miR-127 | MIMAT0000139 | ucggauccgucugagcuuggcu | 68 |
| mmu-miR-434-3p | MIMAT0001422 | uuugaaccaucacucgacuccu | 70 |
| mmu-miR-434-5p | MIMAT0001421 | gcucgacucaugguuugaacca | 71 |
| mmu-miR-136 | MIMAT0000148 | acuccauuuguuuugaugaugg | 73 |
| mmu-miR-136* | MIMAT0004532 | aucaucgucucaaaugagucuu | 74 |
| mmu-miR-341 | MIMAT0000588 | ucggucgaucggucggucggu | 75 |
| mmu-miR-379 | MIMAT0000743 | ugguagacuauggaacguagg | 79 |
| mmu-miR-411 | MIMAT0004747 | uaguagaccguauagcguacg | 80 |
| mmu-miR-411* | MIMAT0001093 | uauguaacacgguccacuaacc | 81 |
| mmu-miR-299* | MIMAT0000377 | ugguuuaccgucccacauacau | 83 |
| mmu-miR-380-3p | MIMAT0000745 | uauguaguaugguccacaucuu | 84 |
| mmu-miR-323-3p | MIMAT0000551 | cacauuacacggucgaccucu | 87 |
| mmu-miR-329 | MIMAT0000567 | aacacacccagcuaaccuuuuu | 90 |
| mmu-miR-543 | MIMAT0003168 | aaacauucgcggugcacuucuu | 96 |
| mmu-miR-495 | MIMAT0003456 | aaacaaacauggugcacuucuu | 97 |
| mmu-miR-376c | MIMAT0003183 | aacauagaggaaauuucacgu | 99 |
| mmu-miR-376b | MIMAT0001092 | aucauagaggaacauccacuu | 103 |
| mmu-miR-376b* | MIMAT0003388 | auggauauuccuucuaugguua | 104 |
| mmu-miR-376a | MIMAT0000740 | aucguagaggaaaauccacgu | 105 |
| mmu-miR-300 | MIMAT0000378 | uaugcaagggcaagcucucuuc | 107 |
| mmu-miR-381 | MIMAT0000746 | uauacaagggcaagcucucugu | 109 |
| mmu-miR-487b | MIMAT0003184 | aaucguacagggucauccacuu | 110 |
| mmu-miR-382 | MIMAT0000747 | gaaguuguucgugguggauucg | 113 |
| mmu-miR-382* | MIMAT0004691 | ucauucacggacaacacuuuuu | 114 |
| mmu-miR-154 | MIMAT0000164 | uagguuauccguguugccuucg | 120 |
| mmu-miR-154* | MIMAT0004537 | aaucauacacgguugaccuauu | 121 |
| mmu-miR-377 | MIMAT0000741 | aucacacaaaggcaacuuuugu | 122 |
| mmu-miR-541 | MIMAT0003170 | aagggauucugauguuggucacacu | 124 |
| mmu-miR-409-3p | MIMAT0001090 | gaauguugcucggugaaccccu | 125 |
| mmu-miR-409-5p | MIMAT0004746 | agguuacccgagcaacuuugcau | 126 |
| mmu-miR-369-3p | MIMAT0003186 | aauaauacaugguugaucuuu | 128 |
| mmu-miR-369-5p | MIMAT0003185 | agaucgaccguguuauauucgc | 129 |
| mmu-miR-410 | MIMAT0001091 | aauauaacacagauggccugu | 130 |

Confirmation of microRNA Expression in Human Cells

Profiling of the expression of microRNA expressed in cells shown in Table 8 was performed using Human miRNA microarray V3 (Agilent).

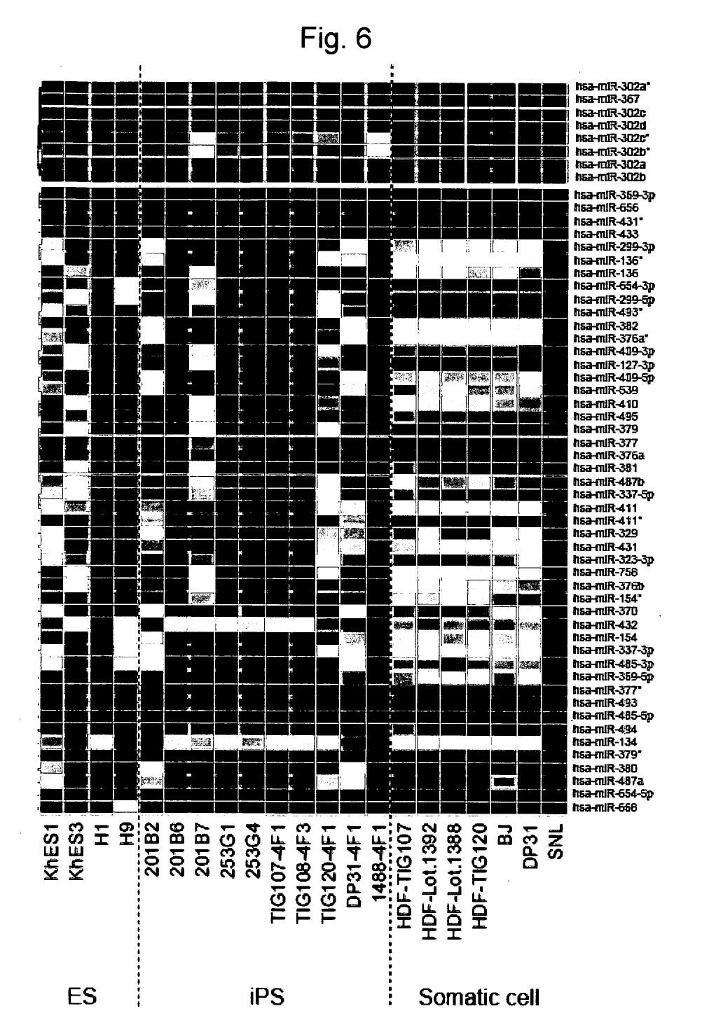

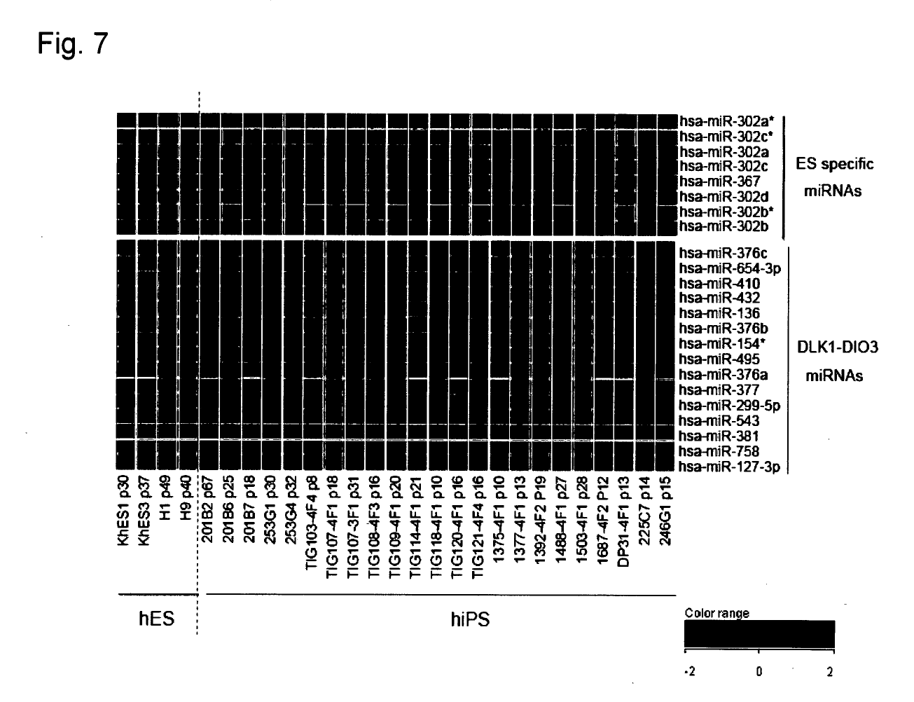

The results of several probes of Group III human miRNA not expressed in somatic cells but expressed in ES cells and iPS cells and Group IV human miRNA of Dlk1-Dio3 region were are shown in FIGS. 6 and 7. The list of Group III is shown Table 11 and the list of Group IV is shown in Table 12.

A lot of Group IV human miRNA was expressed in ES cell clones (KhES1 and KhES3) and iPS cell clones (201B2, 201B7, TIG103-4F4, TIG114-4F1, TIG120-4F1, 1375-4F1, 1687-4F2 and DP31).

TABLE 11
Group III human miRNA

| | | | SEQ |
|---|---|---|---|
| | | | ID |
| ID | Accession | Sequence | NO: |
| hsa-miR-302a* | MIMAT0000683 | acuuaaacguggauguacuug | 262 |
| | | cu | |
| hsa-miR-367 | MIMAT0000719 | aauugcacuuuagcaauggu | 263 |
| | | ga | |
| hsa-miR-302c | MIMAT0000717 | uaagugcuuccauguuucagu | 264 |
| | | gg | |
| hsa-miR-302d | MIMAT0000718 | uaagugcuuccauguuugagu | 265 |
| | | gu | |
| hsa-miR-302c* | MIMAT0000716 | uuuaacauggggguaccugc | 266 |
| | | ug | |
| hsa-miR-302b* | MIMAT0000714 | acuuuaacauggaagugcuu | 267 |
| | | uc | |
| hsa-miR-302a | MIMAT0000684 | uaagugcuuccauguuuuggu | 268 |
| | | ga | |
| hsa-miR-302b | MIMAT0000715 | uaagugcuuccauguuuuagu | 269 |
| | | ag | |

TABLE 12
Group IV human miRNA

| | | | SEQ |
|---|---|---|---|
| | | | ID |
| ID | Accession | Sequence | NO: |
| hsa-miR-369- | MIMAT0000721 | aauaauacaugguugaucuuu | 247 |
| 3p | | | |
| hsa-miR-656 | MIMAT0003332 | aauauuauacagucaaccucu | 249 |
| hsa-miR-431* | MIMAT0004757 | caggucgucuugcagggcuu | 189 |
| | | cu | |
| hsa-miR-433 | MIMAT0001627 | aucaugaugggcuccucggu | 190 |
| | | gu | |
| hsa-miR-299- | MIMAT0000687 | uaugugggaugguaaaccgc | 203 |
| 3p | | uu | |
| hsa-miR-136* | MIMAT0004606 | caucaucgucucaaaugagu | 196 |
| | | cu | |
| hsa-miR-136 | MIMAT0000448 | acuccauuuguuuugaugau | 195 |
| | | gga | |
| hsa-miR-654- | MIMAT0004814 | uaugucugcugaccaucacc | 217 |
| 3p | | uu | |
| hsa-miR-299- | MIMAT0002890 | ugguuuaccgucccacauac | 202 |
| 5p | | au | |
| hsa-miR-493* | MIMAT0002813 | uuguacaugguaggcuuuca | 184 |
| | | uu | |
| hsa-miR-382 | MIMAT0000737 | gaaguuguucgugguggauu | 230 |
| | | cg | |
| hsa-miR-376a* | MIMAT0003386 | guagauucuccuucuaugag | 220 |
| | | ua | |
| hsa-miR-409- | MIMAT0001639 | gaauguugcucggugaaccc | 244 |
| 3p | | cu | |
| hsa-miR-127- | MIMAT0000446 | ucggauccgucugagcuugg | 192 |
| 3p | | cu | |
| hsa-miR-409- | MIMAT0001638 | agguuacccgagcaacuuug | 243 |
| 5p | | cau | |
| hsa-miR-539 | MIMAT0003163 | ggagaaauuauccuuggugu | 225 |
| | | gu | |
| hsa-miR-410 | MIMAT0002171 | aauauaacacagauggccugu | 248 |
| hsa-miR-495 | MIMAT0002817 | aaacaaacauggugcacuuc | 213 |
| | | uu | |
| hsa-miR-379 | MIMAT0000733 | ugguagacuauggaacguagg | 198 |
| hsa-miR-377 | MIMAT0000730 | aucacacaaaggcaacuuuu | 239 |
| | | gu | |
| hsa-miR-376a | MIMAT0000729 | aucauagaggaaaauccacgu | 219 |
| hsa-miR-381 | MIMAT0000736 | uauacaagggcaagcucucu | 223 |
| | | gu | |
| hsa-miR-487b | MIMAT0003180 | aaucguacagggucauccac | 224 |
| | | uu | |
| hsa-miR-337- | MIMAT0004695 | gaacggcuucauacaggaguu | 185 |
| 5p | | | |
| hsa-miR-411 | MIMAT0003329 | uaguagaccguauagcguacg | 200 |
| hsa-miR-411* | MIMAT0004813 | uauguaacacgguccacuaa | 201 |
| | | cc | |
| hsa-miR-329 | MIMAT0001629 | aacacaccugguuaaccucu | 210 |
| | | uu | |
| hsa-miR-431 | MIMAT0001625 | ugucuugcaggccgucaugca | 188 |
| hsa-miR-323- | MIMAT0000755 | cacauuacacggucgaccucu | 208 |
| 3p | | | |
| hsa-miR-758 | MIMAT0003879 | uuugugaccugguccacuaa | 209 |
| | | cc | |
| hsa-miR-376b | MIMAT0002172 | aucauagaggaaaauccaug | 218 |
| | | uu | |
| hsa-miR-154* | MIMAT0000453 | aaucauacacgguugaccua | 238 |
| | | uu | |
| hsa-miR-370 | MIMAT0000722 | gccugcugggguggaaccug | 197 |
| | | gu | |
| hsa-miR-432 | MIMAT0002814 | ucuuggaguaggucauuggg | 193 |
| | | ugg | |
| hsa-miR-154 | MIMAT0000452 | uagguuauccguguugccuu | 236 |
| | | cg | |
| hsa-miR-337- | MIMAT0000754 | cuccuauaugaugccuuucu | 186 |
| 3p | | uc | |
| hsa-miR-485- | MIMAT0002176 | gucauacacggcucuccucu | 234 |
| 3p | | cu | |
| hsa-miR-369- | MIMAT0001621 | agaucgaccguguuauauuc | 246 |
| 5p | | gc | |
| hsa-miR-377* | MIMAT0004689 | agagguugcccuuggugaau | 240 |
| | | uc | |
| hsa-miR-493 | MIMAT0003161 | ugaaggucuacugugugcca | 183 |
| | | gg | |
| hsa-miR-485- | MIMAT0002175 | agaggcuggccgugaugaau | 233 |
| 5p | | uc | |
| hsa-miR-494 | MIMAT0002816 | ugaaacauacacgggaaacc | 211 |
| | | uc | |
| hsa-miR-134 | MIMAT0000447 | ugugacugguugaccagagg | 231 |
| | | gg | |
| hsa-miR-379* | MIMAT0004690 | uauguaacaugguccacuaa | 199 |
| | | cu | |
| hsa-miR-380 | MIMAT0000735 | uauguaauaugguccacauc | 204 |
| | | uu | |
| hsa-miR-487a | MIMAT0002178 | aaucauacagggacauccag | 229 |
| | | uu | |
| hsa-miR-654- | MIMAT0003330 | uggugggccgcagaacaugu | 216 |
| 5p | | gc | |
| hsa-miR-668 | MIMAT0003881 | ugucacucggcucggcccac | 232 |
| | | uac | |
| hsa-miR-376c | MIMAT0000720 | aacauagaggaaauuccac | 214 |
| | | gu | |
| hsa-miR-543 | MIMAT0004954 | aaacauucgcggugcacuuc | 212 |
| | | uu | |

Confirmation of Mouse mRNA Expression of Dlk1, Meg3/Gtl2, Meg8/Rian, Meg9/Mirg, and Dio3 Gene

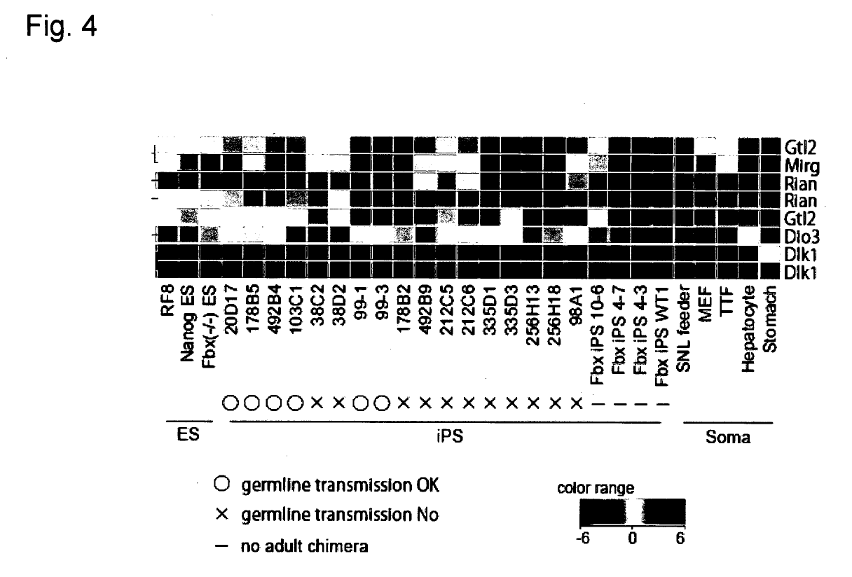

Expression of Dlk1, Meg3/Gtl2, Meg8/Rian, Meg9/Mirg, and Dio3 encoded by the same gene sites as in the case of the above Group II miRNA was examined using gene expression arrays (Agilent). The results are shown in FIG. 4. The Dlk1 gene and Dio3 gene that are expressed only in a paternally derived chromosome were expressed in almost the same manner among iPS cells clones. However, Meg3/Gtl2, Meg8/Rian, and Meg9/Mirg genes that are expressed only in a maternally derived chromosome were expressed in various manners among iPS cell clones and the distribution of the expression correlated with that for Group II miRNA above. Therefore, it was suggested that the genes that are expressed only in a maternally derived chromosome are useful as markers for iPS cells having functions equivalent to those of ES cells in which germline transmission occurs.

Confirmation of Human mRNA Expression of MEG3 and MEG8 Gene

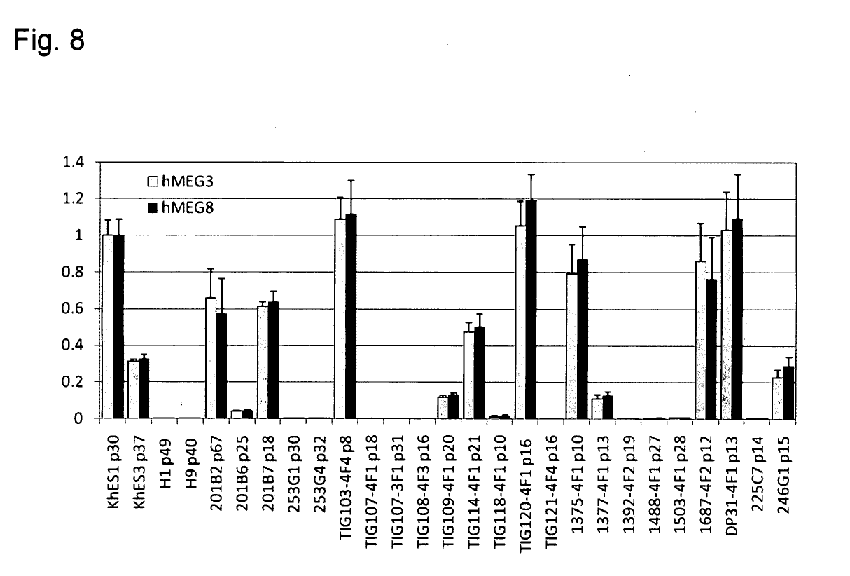

Expression of MEG3 mRNA and MEG8 mRNA in ES cells and iPS cells was examined using Quantitative-PCR (qPCR) by Taqman probe whose assay ID of MEG3, MEG8 and GAPDH as internal standard were respectively Hs00292028_m1, Hs00419701_m1 and Hs03929097_g1 (Applied biosystems). The results are shown in FIG. 8. KhES1, 201B2, 201B7, TIG103-4F4, TIG114-4F1, TIG120-4F1, 1375-4F1, 1687-4F2 and DP31-4F1 were highly expressing these genes. Thus, these genes expression were correlated with the expression of miRNA located in human DLK1-DIO3 region shown in FIGS. 6 and 7.

### Confirmation of DNA Methylation of IG-DMR and MEG3-DMR

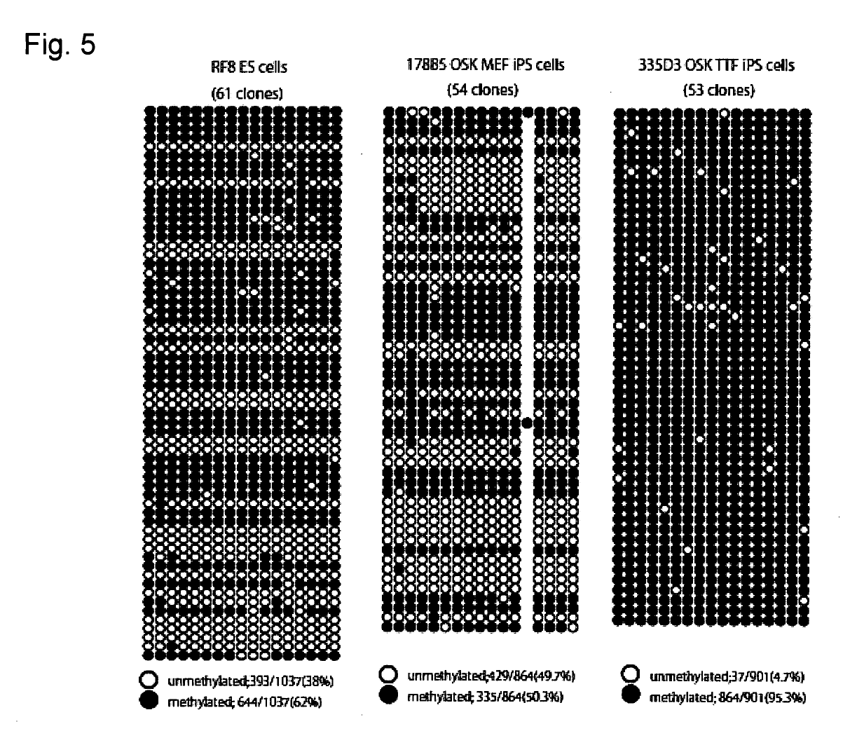

Methylation of IG-DMR (see Cytogenet Genome Res 113: 223-229, (2006)) was examined for germline-competent mouse iPS cells (178B5) prepared by introducing 3 genes (OSK) into MEF, ES cells (RF8) and iPS cells (335D3) prepared by introducing 3 genes (OSK) into TTF for which no germline transmission had been confirmed. Specifically, DNA methylation of the CG sequence in a 351-bp portion ranging from nucleotide 80479 to nucleotide 80829 in the AJ320506 sequence (NCBI) was measured. DNA methylation was confirmed by treating DNA extracted from subject cells using a MethylEasy Xceed Rapid DNA Bisulphite Modification Kit (Human genetics) as a reagent for bisulfite treatment, amplifying IG-DMR by PCR, and then analyzing the cloned PCR products using a capillary sequencer. The experiment was conducted a plurality of times. One of the results is shown in FIG. 5. In the case of ES cells (RF8), 62% of 61 clones measured were methylated; and in the case of 178B5 iPS cells, 50% of 54 clones measured were methylated. This is inferred to be a state in which either a paternally-derived or a maternally-derived chromosome alone was methylated. Hence, it is considered that normal imprinting was carried out in these two cell lines. On the other hand, in the case of 335D3 iPS cells in which no germline transmission occurs, results indicating abnormal imprinting (e.g., when all CpG cytosines had been methylated) were obtained. Accordingly, it was suggested that iPS cells in which germline transmission occurs can be screened for by measuring IG-DMR methylation and then confirming if imprinting of the region is normal or not.

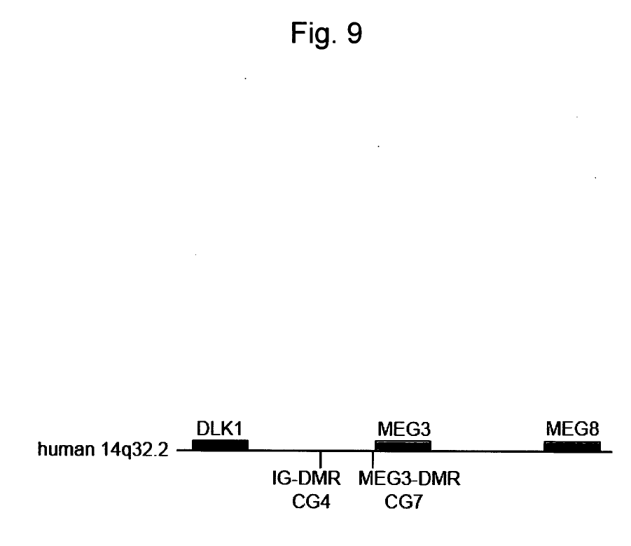

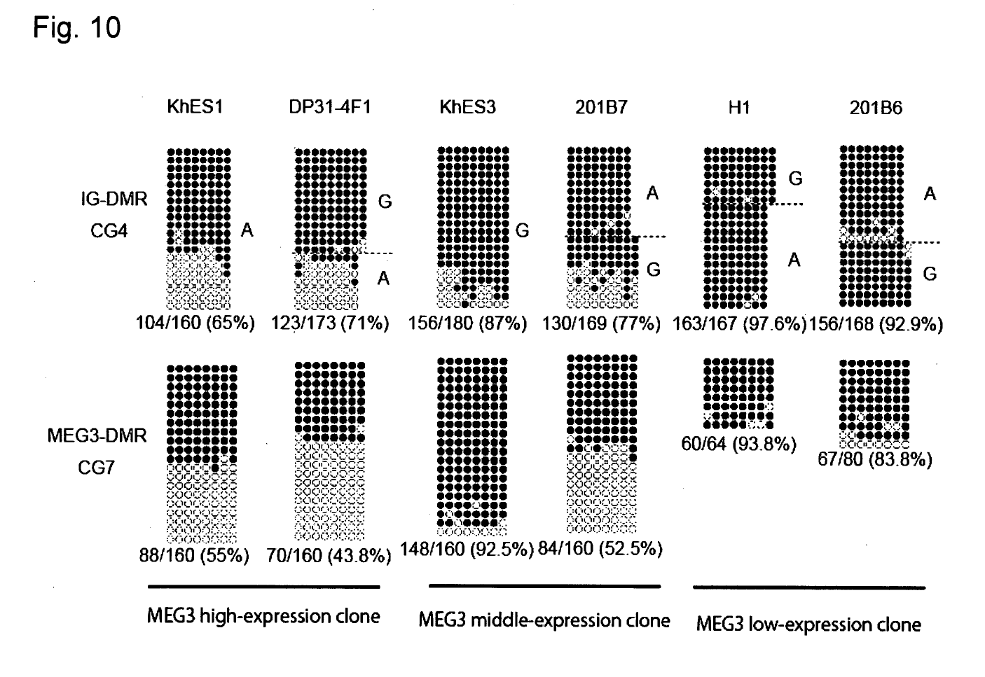

Similarly, the concentration of methylated cytosine in IG-DMR CG4 and MEG3-DMR CG7 shown in FIG. 9 was examined in human cells. The result of each clones (KhES1, DP31-4F1, KhES3, 201B7, H1 and 201B6) is shown in FIG. 10, wherein KhES1 and DP31-4F1 were exemplified as the high MEG3 expression clones, KhES3 and 20187 as middle MEG3 expression clones and H1 and 201B6 as low MEG3 expression clones according to result of qPCR shown in FIG. 8. The degree of DNA methylation in IG-DMR CG4 and MEG3-DMR CG7 was inversely-correlating with the expression of MEG3 and MEG8 mRNA. For example, 65% cytosines in IG-DMR CG4 were methylated in IG-DMR of DP31-4F1 which was highly expressing MEG3 and MEG8 mRNA. On the contrary, 93% cytosine in IG-DMR CG4 were methylated in IG-DMR of 201B6 which less expressed MEG3 and MEG8 mRNA.

Meanwhile, it was examined whether undifferentiated cells expressing Oct-3/4 genes were include in the differentiated neural cells from each ES cells or iPS cells using SFEBq method. The said SFEBq method was performed with method comprising following steps of:

(i) the ES cells or iPS cells were cultured with medium containing Y27632;

(ii) for removal of feeder cells CTK dissociation solution (0.25% Trypsin, 1 mg/ml Collagenase and KSR 20%, and 1 mM CaCl2) was added to culture dish and transfer to gelatin coated dish;

(iii) the ES cells or iPS cells were dissociated with Accumax™;

(iv) the dissociated ES cells or iPS cells were transfer to LIPIDURE-COAT PLATE (NOF Corporation) and cultured with differentiation medium (DMEM/Ham's F12 containing 5% knockout serum replacement (KSR), 2 mM L-glutamine, non-essential amino acids, and 1 micro-M 2-mercaptoethanol (2-ME)) contained 10 micro-M Y27632, 2 micro-M Dorsomorphin (Sigma) and 10 micro-M SB431542 (Sigma) for 3 or 4 days; and

(v) Half media was changed with new differentiation medium without Y27632, Dorsomorphin and SB431542 and cultured for more 10 or 11 days.

After the differentiation to neural cells, clones of TIG108-4F3 (relative value of MEG3 and MEG8 mRNA expression shown in FIG. 8 are 0 and 0.00083) and TIG118-4F1 (relative value of MEG3 and MEG8 mRNA expression shown in FIG. 8 are 0.012 and 0.017) still included Oct3/4 positive cells when checking by flow cytometer. On the contrary, clones of KhES1, 201B7 (relative value of MEG3 and MEG8 mRNA expression shown in FIG. 8 are 0.61 and 0.64) and so on included no Oct3/4 positive cells.

These result showed that degree of DNA methylated in IG-DMR and MEG3-DMR and expression of MEG3 and/or MEG8 were able to be used as the marker of quality (e.g. pluripotency and ability for easy induction of differentiation) of iPS cells.

1. A method for screening an induced pluripotent stem cell(s), comprising the following steps of:
(1) measuring the expression level of at least one miRNA or gene located in an imprinted region in a subject induced pluripotent stem cell(s); and,
(2) selecting the induced pluripotent stem cell(s) expressing the miRNA or the gene at a level equivalent to or higher than that of a control cell(s).

2. The method according to claim 1, wherein the imprinted region is a Dlk1-Dio3 region.

3. The method according to claim 1, wherein the miRNA is selected from the group consisting of the pri-miRNA shown in Tables 1 and 3 and the mature-miRNA shown in Tables 2 and 4.

4. The method according to claim 1, wherein the gene is selected from the group consisting of the genes shown in Table 5.

5. The method according to claim 4, wherein the gene is selected from the group consisting of MEG3 and MEG8.

6. The method according to claim 1, wherein the control cell(s) is/are an embryonic stem cell(s).

7. A method for screening induced pluripotent stem cells, comprising the following steps of:
(1) measuring a DNA methylation state in an imprinted region of a subject induced pluripotent stem cell(s); and
(2) selecting the induced pluripotent stem cell(s) in which the imprinted region in a/one chromosome is in a DNA-methylated state, but the same region in a homologous chromosome is not in a DNA-methylated state.

8. The method according to claim 7, wherein the imprinted region is IG-DMR and/or Gtl2/MEG3-DMR.

9. The method according to claim 7, comprising the step of selecting an induced pluripotent stem cell(s) in which the imprinted region in a paternally-derived chromosome is in the DNA-methylated state.

10. The method according to claim 1 or 9, wherein the induced pluripotent stem cell(s) is/are capable of germline transmission.

11. A kit for screening induced pluripotent stem cells, which comprises at least one primer set or probe for detecting pri-miRNA shown in Table 1 or 3, miRNA shown in Table 2 or 4, and a gene shown in Table 5.

12. The kit according to claim 11, which comprises a microarray.

13. A kit for screening induced pluripotent stem cells, which comprises a methylation-sensitive restriction enzyme, or a bisulfate reagent and a nucleic acid for amplification of IG-DMR and/or Gtl2/MEG3-DMR.

14. An induced pluripotent stem cell capable of germline transmission, which is screened for by the method according to claim 1.


SEQUENCE LISTING (275 sequences)

SEQ ID NO: 1 - RNA, 94 nt, Mus musculus
gccaccuucugugcccccagcaccacgugucugggccacgugagcaacgccacgugggccugacguggagcuggggccgcaggggucugauggc

SEQ ID NO: 2 - RNA, 91 nt, Mus musculus
uggagccugaggggcucacagcucugguccuuggagcuccagagaaaauguugcuccggggcugaguucugugcaccccccuugcccucca

SEQ ID NO: 3 - RNA, 83 nt, Mus musculus
cgccagggccuuguacaugguaggcuuucauucauuuuuugcacauucggugaagguccuacugugugccaggcccugugcca

SEQ ID NO: 4 - RNA, 97 nt, Mus musculus
caguguagugagaaguuggggggugggaacggcgucaugcaggaguugauugcacagccauucagcuccuauaugaugccuuucuucacccccuuca

SEQ ID NO: 5 - RNA, 67 nt, Mus musculus
ugggccaagggucacccucugacucuguggccaaggguagacaggucagaggucgauccugggccua

SEQ ID NO: 6 - RNA, 94 nt, Mus musculus
agaacagggucuccuugaggggccucugccucuauccaggauuauguuuuuaugaccaggaggcugaggucccuuacaggcggccucuuacucu

SEQ ID NO: 7 - RNA, 91 nt, Mus musculus
cguccugcgaggugucuugcaggccgucaugcaggccacacugacgguaacguugcaggucgucuugcagggcuucucgcaagacgacauc

SEQ ID NO: 8 - RNA, 124 nt, Mus musculus
ugcccggggagaaguacggugagccugucauuauucagagaggcuagauccucuguguugagaaggaucaugaugggcuccucgguguucuccagguagcggcaccacaccaugaaggcagccc

SEQ ID NO: 9 - RNA, 70 nt, Mus musculus
ccagccugcugaagcucagagggcucugauucagaaagaucaucggauccgucugagcuuggcuggucgg

SEQ ID NO: 10 - RNA, 94 nt, Mus musculus
ucgacucuggguuugaaccaaagcucgacucaugguuugaaccauuacuuaauucgugguuugaaccaucacucgacuccugguucgaaccauc

SEQ ID NO: 11 - RNA, 75 nt, Mus musculus
uggguagcucuugcauuuccuggugggggccacuggauggcuccuccacuucuuggaguagaucagugggcagcu

SEQ ID NO: 12 - RNA, 62 nt, Mus musculus
gaggacuccauuuguuuugaugauggauucuuaagcuccaucaucgucucaaaugagucuuc

SEQ ID NO: 13 - RNA, 96 nt, Mus musculus
aaaaugaugaugucaguuggccggucggccgaucgcucggucugucagucagucggucggucgaucggucggucggucagucggcuuccugucuuc

SEQ ID NO: 14 - RNA, 120 nt, Mus musculus
auacucacagucucccagcuggugugagguugggccaggaugaaacccaaggcucuccgaggcuccccaccacacccugcugcugaagacugccuagcaaggcugugccgaguggugugg

SEQ ID NO: 15 - RNA, 79 nt, Mus musculus
agacggagagaccaggucacgucucugcaguuacacagcucaugagugccugcugggguggaaccugguuugucugucu

SEQ ID NO: 16 - RNA, 77 nt, Mus musculus
cagcaguaccaggagagaguuagcgcauuagugcaauaguuaguccugauuucuggguuuuucuaauggcugcucuu

SEQ ID NO: 17 - RNA, 66 nt, Mus musculus
agagaugguagacuauggaacguaggcguuauguuuuugaccuauguaacaugguccacuaacucu

SEQ ID NO: 18 - RNA, 82 nt, Mus musculus
ugguacuuggagagauaguagaccguauagcguacgcuuuaucugugacguauguaacacgguccacuaacccucaguauca

SEQ ID NO: 19 - RNA, 63 nt, Mus musculus
aagaaaugguuuaccgucccacauacauuuugaguauguaugugggacgguaaaccgcuucuu

SEQ ID NO: 20 - RNA, 61 nt, Mus musculus
aagaugguugaccauagaacaugcgcuacuucugugucguauguaguaugguccacaucuu

SEQ ID NO: 21 - RNA, 120 nt, Mus musculus
gugagcuggaaucagccagcguuaccucaagguauuugaagaugcgguugaccaugguguguacgcuuuauuuaugacguaggacacauggucuacuucuucucaauaucacaucucgcc

SEQ ID NO: 22 - RNA, 86 nt, Mus musculus
uugguacuuggagagaggugguccguggcgcguucgcuucauuuauggcgcacauuacacggucgaccucuuugcgguaucuaauc

SEQ ID NO: 23 - RNA, 81 nt, Mus musculus
ugggugcgugaggugguugaccagagagcacacgcuauauuugugccguuugugaccugguccacuaacccucaguaucua

SEQ ID NO: 24 - RNA, 97 nt, Mus musculus
uguucgcuucugguaccggaagagagguuuucugggucucuguuucuuugaugagaaugaaacacacccagcuaaccuuuuuuucaguaucaaaucc

SEQ ID NO: 25 - RNA, 85 nt, Mus musculus
uugauacuugaaggagagguuguccguguugucuucucuuuauuuaugaugaaacauacacgggaaaccucuuuuuuaguaucaa

SEQ ID NO: 26 - RNA, 74 nt, Mus musculus
cuauggcuuuggacugugaggugacucuuggugugugauggcuuuucagcaagguccuccucacaguagcuaua

SEQ ID NO: 27 - RNA, 121 nt, Mus musculus
cugaagggacaaugaugcccacuguucucgggguagcuguguggaugguagaccggugacguacacuucauuuaugcuguaggucacccguuuuacuauccaccaacacccagaccaucug

SEQ ID NO: 28 - RNA, 99 nt, Mus musculus
cugauucugccugcguggagcgggcacagcugugagagcccccuagguacagcggggcugcagcgugaucgccugcucacgcacaggaagugacgacag

SEQ ID NO: 29 - RNA, 76 nt, Mus musculus
ugcuuaaugagaaguugcccgcguguuuuucgcuuuauaugugacgaaacauucgcggugcacuucuuuuucagca

SEQ ID NO: 30 - RNA, 63 nt, Mus musculus
aaagaaguugcccauguuauuuuucgcuuuuauuugugacgaaacaaacauggugcacuucuu

SEQ ID NO: 31 - RNA, 92 nt, Mus musculus
guggguacuggccucggugcugguggagcagugagcacgccauacauuauaucugugacaccugccacccagcccaaggccccuaggcccac

SEQ ID NO: 32 - RNA, 86 nt, Mus musculus
uuugguauuuaaaagguggauauuccuucuauguuuaugcuuuuugugauuaaacauagaggaaauuucacguuuucagugucaaa

SEQ ID NO: 33 - RNA, 84 nt, Mus musculus
cucgguaagugggaagaugguaagcugcagaacauguguguuucucaugucauaugucugcugaccaucaccuuugggucucug

SEQ ID NO: 34 - RNA, 82 nt, Mus musculus
ugguauuuaaaagguggauauuccuucuaugguuacgugcuuccuggauaaucauagaggaacauccacuuuuucaguauca

SEQ ID NO: 35 - RNA, 68 nt, Mus musculus
uaaaagguagauucuccuucuaugaguacaauauuaaugacuaaucguagaggaaaauccacguuuuc

SEQ ID NO: 36 - RNA, 79 nt, Mus musculus
gcuacuugaagagagguuauccuuuguguguuugcuuuacgcgaaaugaauaugcaagggcaagcucucuucgaggagc

SEQ ID NO: 37 - RNA, 75 nt, Mus musculus
uacuuaaagcgagguugcccuuuguauauucgguuuauugacauggaauauacaagggcaagcucucugugagua

SEQ ID NO: 38 - RNA, 82 nt, Mus musculus
ugguacuuggagagugguuaucccuguccucuucgcuucacucaugccgaaucguacagggucauccacuuuuucaguauca

SEQ ID NO: 39 - RNA, 74 nt, Mus musculus
uacuugaggagaaauuauccuugguguguuggcucuuuuggaugaaucauacaaggauaauuucuuuuugagua

SEQ ID NO: 40 - RNA, 78 nt, Mus musculus
caccuagggaucuuguuaaaaagcagagucugauugaggggccaagauucugcauuuuuagcaagcucucaagugaug

SEQ ID NO: 41 - RNA, 76 nt, Mus musculus
uacuugaagagaaguuguucgugguggauucgcuuuacuugugacgaaucauucacggacaacacuuuuuucagua

SEQ ID NO: 42 - RNA, 71 nt, Mus musculus
agggugugugacugguugaccagaggggcgugcacucuguucacccugugggccaccuagucaccaacccu

SEQ ID NO: 43 - RNA, 66 nt, Mus musculus
gguaagugugccucgggugagcaugcacuuaauguagguguaugucacucggcucggcccacuacc

SEQ ID NO: 44 - RNA, 73 nt, Mus musculus
acuuggagagaggcuggccgugaugaauucgauucaucuaaacgagucauacacggcucuccucucuucuagu

SEQ ID NO: 45 - RNA, 82 nt, Mus musculus
agaagaugcaggagugcugugagaagugccauccccugguacuuggagggagguugccucauagugagcuugcauuauuuaa

SEQ ID NO: 46 - RNA, 66 nt, Mus musculus
gaagauagguuauccguguugccuucgcuuuauucgugacgaaucauacacgguugaccuauuuuu

SEQ ID NO: 47 - RNA, 79 nt, Mus musculus
aguguucgaauggagguugcccaugguguguucauuuuauuuaugaugaguauuacauggccaaucuccuuucggcacu

SEQ ID NO: 48 - RNA, 68 nt, Mus musculus
ugagcagagguugcccuuggugaauucgcuuuauugauguugaaucacacaaaggcaacuuuuguuug

SEQ ID NO: 49 - RNA, 90 nt, Mus musculus
gccaaaaucagagaagggauucugauguuggucacacuccaagaguuuuaaaaugaguggcgaacacagaauccauacucugcuuauggc

SEQ ID NO: 50 - RNA, 79 nt, Mus musculus
ugguacucggagagagguuacccgagcaacuuugcaucuggaggacgaauguugcucggugaaccccuuuucgguauca

SEQ ID NO: 51 - RNA, 80 nt, Mus musculus
ggguaugggacggauggucgaccagcuggaaaguaauuguuucuaauguacuucaccugguccacuagccgucggugccc

SEQ ID NO: 52 - RNA, 79 nt, Mus musculus
gguacuugaagggagaucgaccguguuauauucgcuuggcugacuucgaauaauacaugguugaucuuuucucaguauc

SEQ ID NO: 53 - RNA, 81 nt, Mus musculus
ggguacuugaggagagguugucugugaugaguucgcuuuauuaaugacgaauauaacacagauggccuguuuucaauacca

SEQ ID NO: 54 - RNA, 22 nt, Mus musculus
cgugggccugacguggagcugg

SEQ ID NO: 55 - RNA, 22 nt, Mus musculus
agcaccacgugucugggccacg

SEQ ID NO: 56 - RNA, 23 nt, Mus musculus
uccggggcugaguucugugcacc

SEQ ID NO: 57 - RNA, 22 nt, Mus musculus
cucacagcucugguccuuggag

SEQ ID NO: 58 - RNA, 23 nt, Mus musculus
ugaagguccuacugugugccagg

SEQ ID NO: 59 - RNA, 21 nt, Mus musculus
uucagcuccuauaugaugccu

SEQ ID NO: 60 - RNA, 21 nt, Mus musculus
gaacggcgucaugcaggaguu

SEQ ID NO: 61 - RNA, 20 nt, Mus musculus
aggucagaggucgauccugg

SEQ ID NO: 62 - RNA, 23 nt, Mus musculus
caagggucacccucugacucugu

SEQ ID NO: 63 - RNA, 20 nt, Mus musculus
accaggaggcugaggucccu

SEQ ID NO: 64 - RNA, 21 nt, Mus musculus
ugucuugcaggccgucaugca

SEQ ID NO: 65 - RNA, 22 nt, Mus musculus
caggucgucuugcagggcuucu

SEQ ID NO: 66 - RNA, 22 nt, Mus musculus
aucaugaugggcuccucggugu

SEQ ID NO: 67 - RNA, 22 nt, Mus musculus
uacggugagccugucauuauuc

SEQ ID NO: 68 - RNA, 22 nt, Mus musculus
ucggauccgucugagcuuggcu

SEQ ID NO: 69 - RNA, 22 nt, Mus musculus
cugaagcucagagggcucugau

SEQ ID NO: 70 - RNA, 22 nt, Mus musculus
uuugaaccaucacucgacuccu

SEQ ID NO: 71 - RNA, 22 nt, Mus musculus
gcucgacucaugguuugaacca

SEQ ID NO: 72 - RNA, 23 nt, Mus musculus
ucuuggaguagaucagugggcag

SEQ ID NO: 73 - RNA, 22 nt, Mus musculus
acuccauuuguuuugaugaugg

SEQ ID NO: 74 - RNA, 22 nt, Mus musculus
aucaucgucucaaaugagucuu

SEQ ID NO: 75 - RNA, 21 nt, Mus musculus
ucggucgaucggucggucggu

SEQ ID NO: 76 - RNA, 21 nt, Mus musculus
uggugugagguugggccagga

SEQ ID NO: 77 - RNA, 22 nt, Mus musculus
gccugcugggguggaaccuggu

SEQ ID NO: 78 - RNA, 22 nt, Mus musculus
aggagagaguuagcgcauuagu

SEQ ID NO: 79 - RNA, 21 nt, Mus musculus
ugguagacuauggaacguagg

SEQ ID NO: 80 - RNA, 21 nt, Mus musculus
uaguagaccguauagcguacg

SEQ ID NO: 81 - RNA, 22 nt, Mus musculus
uauguaacacgguccacuaacc

SEQ ID NO: 82 - RNA, 22 nt, Mus musculus
uaugugggacgguaaaccgcuu

SEQ ID NO: 83 - RNA, 22 nt, Mus musculus
ugguuuaccgucccacauacau

SEQ ID NO: 84 - RNA, 22 nt, Mus musculus
uauguaguaugguccacaucuu

SEQ ID NO: 85 - RNA, 22 nt, Mus musculus
augguugaccauagaacaugcg

SEQ ID NO: 86 - RNA, 21 nt, Mus musculus
uaggacacauggucuacuucu

SEQ ID NO: 87 - RNA, 21 nt, Mus musculus
cacauuacacggucgaccucu

SEQ ID NO: 88 - RNA, 22 nt, Mus musculus
aggugguccguggcgcguucgc

SEQ ID NO: 89 - RNA, 19 nt, Mus musculus
uuugugaccugguccacua

SEQ ID NO: 90 - RNA, 22 nt, Mus musculus
aacacacccagcuaaccuuuuu

SEQ ID NO: 91 - RNA, 22 nt, Mus musculus
ugaaacauacacgggaaaccuc

SEQ ID NO: 92 - RNA, 22 nt, Mus musculus
ggacugugaggugacucuuggu

SEQ ID NO: 93 - RNA, 21 nt, Mus musculus
uaggucacccguuuuacuauc

SEQ ID NO: 94 - RNA, 22 nt, Mus musculus
ggcugcagcgugaucgccugcu

SEQ ID NO: 95 - RNA, 22 nt, Mus musculus
agcgggcacagcugugagagcc

SEQ ID NO: 96 - RNA, 22 nt, Mus musculus
aaacauucgcggugcacuucuu

SEQ ID NO: 97 - RNA, 22 nt, Mus musculus
aaacaaacauggugcacuucuu

SEQ ID NO: 98 - RNA, 23 nt, Mus musculus
ugacaccugccacccagcccaag

SEQ ID NO: 99 - RNA, 21 nt, Mus musculus
aacauagaggaaauuucacgu

SEQ ID NO: 100 - RNA, 22 nt, Mus musculus
guggauauuccuucuauguuua

SEQ ID NO: 101 - RNA, 22 nt, Mus musculus
uaugucugcugaccaucaccuu

SEQ ID NO: 102 - RNA, 22 nt, Mus musculus
ugguaagcugcagaacaugugu

SEQ ID NO: 103 - RNA, 21 nt, Mus musculus
aucauagaggaacauccacuu

SEQ ID NO: 104 - RNA, 22 nt, Mus musculus
guggauauuccuucuaugguua

SEQ ID NO: 105 - RNA, 21 nt, Mus musculus
aucguagaggaaaauccacgu

SEQ ID NO: 106 - RNA, 22 nt, Mus musculus
gguagauucuccuucuaugagu

SEQ ID NO: 107 - RNA, 22 nt, Mus musculus
uaugcaagggcaagcucucuuc

SEQ ID NO: 108 - RNA, 22 nt, Mus musculus
uugaagagagguuauccuuugu

SEQ ID NO: 109 - RNA, 22 nt, Mus musculus
uauacaagggcaagcucucugu

SEQ ID NO: 110 - RNA, 22 nt, Mus musculus
aaucguacagggucauccacuu

SEQ ID NO: 111 - RNA, 22 nt, Mus musculus
ggagaaauuauccuuggugugu

SEQ ID NO: 112 - RNA, 22 nt, Mus musculus
auucugcauuuuuagcaagcuc

SEQ ID NO: 113 - RNA, 22 nt, Mus musculus
gaaguuguucgugguggauucg

SEQ ID NO: 114 - RNA, 22 nt, Mus musculus
ucauucacggacaacacuuuuu

SEQ ID NO: 115 - RNA, 22 nt, Mus musculus
ugugacugguugaccagagggg

SEQ ID NO: 116 - RNA, 24 nt, Mus musculus
ugucacucggcucggcccacuacc

SEQ ID NO: 117 - RNA, 22 nt, Mus musculus
agaggcuggccgugaugaauuc

SEQ ID NO: 118 - RNA, 22 nt, Mus musculus
agucauacacggcucuccucuc

SEQ ID NO: 119 - RNA, 24 nt, Mus musculus
agguugccucauagugagcuugca

SEQ ID NO: 120 - RNA, 22 nt, Mus musculus
uagguuauccguguugccuucg

SEQ ID NO: 121 - RNA, 22 nt, Mus musculus
aaucauacacgguugaccuauu

SEQ ID NO: 122 - RNA, 22 nt, Mus musculus
ugaguauuacauggccaaucuc

SEQ ID NO: 123 - RNA, 22 nt, Mus musculus
aucacacaaaggcaacuuuugu

SEQ ID NO: 124 - RNA, 25 nt, Mus musculus
aagggauucugauguuggucacacu

SEQ ID NO: 125 - RNA, 22 nt, Mus musculus
gaauguugcucggugaaccccu

SEQ ID NO: 126 - RNA, 23 nt, Mus musculus
agguuacccgagcaacuuugcau

SEQ ID NO: 127 - RNA, 20 nt, Mus musculus
uucaccugguccacuagccg

SEQ ID NO: 128 - RNA, 21 nt, Mus musculus
aauaauacaugguugaucuuu

SEQ ID NO: 129 - RNA, 22 nt, Mus musculus
agaucgaccguguuauauucgc

SEQ ID NO: 130 - RNA, 21 nt, Mus musculus
aauauaacacagauggccugu

SEQ ID NO: 131 - RNA, 98 nt, Homo sapiens
aggagccaccuuccgagccuccaguaccacgugucagggccacaugagcugggccucgugggccugauguggugcuggggccucaggggucugcucuu

SEQ ID NO: 132 - RNA, 89 nt, Homo sapiens
cuggccuccagggcuuuguacaugguaggcuuucauucauucguuugcacauucggugaaggucuacugugugccaggcccugugccag

SEQ ID NO: 133 - RNA, 93 nt, Homo sapiens
guagucaguaguuggggggugggaacggcuucauacaggaguugaugcacaguuauccagcuccuauaugaugccuuucuucauccccuucaa

SEQ ID NO: 134 - RNA, 72 nt, Homo sapiens
ucuccucgaggggucucugccucuacccaggacucuuucaugaccaggaggcugaggccccucacaggcggc

SEQ ID NO: 135 - RNA, 114 nt, Homo sapiens
uccugcuuguccugcgaggugucuugcaggccgucaugcaggccacacugacgguaacguugcaggucgucuugcagggcuucucgcaagacgacauccucaucaccaacgacg

SEQ ID NO: 136 - RNA, 93 nt, Homo sapiens
ccggggagaaguacggugagccugucauuauucagagaggcuagauccucuguguugagaaggaucaugaugggcuccucgguguucuccagg

SEQ ID NO: 137 - RNA, 97 nt, Homo sapiens
ugugaucacugucuccagccugcugaagcucagagggcucugauucagaaagaucaucggauccgucugagcuuggcuggucggaagucucaucauc

SEQ ID NO: 138 - RNA, 94 nt, Homo sapiens
ugacuccuccaggucuuggaguaggucauuggguggauccucuauuuccuuacgugggccacuggauggcuccuccaugucuuggaguagauca

SEQ ID NO: 139 - RNA, 82 nt, Homo sapiens
ugagcccucggaggacuccauuuguuuugaugauggauucuuaugcuccaucaucgucucaaaugagucuucagaggguucu

SEQ ID NO: 140 - RNA, 75 nt, Homo sapiens
agacagagaagccaggucacgucucugcaguuacacagcucacgagugccugcugggguggaaccuggucugucu

SEQ ID NO: 141 - RNA, 67 nt, Homo sapiens
agagaugguagacuauggaacguaggcguuaugauuucugaccuauguaacaugguccacuaacucu

SEQ ID NO: 142 - RNA, 96 nt, Homo sapiens
ugguacuuggagagauaguagaccguauagcguacgcuuuaucugugacguauguaacacgguccacuaacccucaguaucaaauccauccccgag

SEQ ID NO: 143 - RNA, 63 nt, Homo sapiens
aagaaaugguuuaccgucccacauacauuuugaauauguaugugggaugguaaaccgcuucuu

SEQ ID NO: 144 - RNA, 61 nt, Homo sapiens
aagaugguugaccauagaacaugcgcuaucucugugucguauguaauaugguccacaucuu

SEQ ID NO: 145 - RNA, 88 nt, Homo sapiens
acuuccugguauuugaagaugcgguugaccaugguguguacgcuuuauuugugacguaggacacauggucuacuucuucucaauauca

SEQ ID NO: 146 - RNA, 86 nt, Homo sapiens
uugguacuuggagagaggugguccguggcgcguucgcuuuauuuauggcgcacauuacacggucgaccucuuugcaguaucuaauc

SEQ ID NO: 147 - RNA, 88 nt, Homo sapiens
gccuggauacaugagaugguugaccagagagcacacgcuuuauuugugccguuugugaccugguccacuaacccucaguaucuaaugc

SEQ ID NO: 148 - RNA, 80 nt, Homo sapiens
gguaccugaagagagguuuucuggguuucuguuucuuuaaugaggacgaaacacaccugguuaaccucuuuuccaguauc

SEQ ID NO: 149 - RNA, 84 nt, Homo sapiens
gugguaccugaagagagguuuucuggguuucuguuucuuuauugaggacgaaacacaccugguuaaccucuuuuccaguaucaa

SEQ ID NO: 150 - RNA, 81 nt, Homo sapiens
gauacucgaaggagagguuguccguguugucuucucuuuauuuaugaugaaacauacacgggaaaccucuuuuuuaguauc

SEQ ID NO: 151 - RNA, 78 nt, Homo sapiens
uacuuaaugagaaguugcccguguuuuuuucgcuuuauuugugacgaaacauucgcggugcacuucuuuuucaguauc

SEQ ID NO: 152 - RNA, 82 nt, Homo sapiens
ugguaccugaaaagaaguugcccauguuauuuucgcuuuauaugugacgaaacaaacauggugcacuucuuuuucgguauca

SEQ ID NO: 153 - RNA, 66 nt, Homo sapiens
aaaagguggauauuccuucuauguuuauguuauuuaugguuaaacauagaggaaauuccacguuuu

SEQ ID NO: 154 - RNA, 80 nt, Homo sapiens
gguauuuaaaagguagauuuuccuucuaugguuacguguuugaugguuaaucauagaggaaaauccacguuuucaguauc

SEQ ID NO: 155 - RNA, 81 nt, Homo sapiens
ggguaaguggaaagauggugggccgcagaacaugugcugaguucgugccauaugucugcugaccaucaccuuuagaagccc

SEQ ID NO: 156 - RNA, 100 nt, Homo sapiens
caguccuucuuugguauuuaaaacguggauauuccuucuauguuuacgugauuccugguuaaucauagaggaaaauccauguuuucaguaucaaaugcug

SEQ ID NO: 157 - RNA, 68 nt, Homo sapiens
uaaaagguagauucuccuucuaugaguacauuauuuaugauuaaucauagaggaaaauccacguuuuc

SEQ ID NO: 158 - RNA, 83 nt, Homo sapiens
ugcuacuugaagagagguaauccuucacgcauuugcuuuacuugcaaugauuauacaagggcagacucucucuggggagcaaa

SEQ ID NO: 159 - RNA, 86 nt, Homo sapiens
uuugguacuugaagagaggauacccuuuguauguucacuugauuaauggcgaauauacagggggagacucuuauuugcguaucaaa

SEQ ID NO: 160 - RNA, 86 nt, Homo sapiens
uuugguacuuaaagagaggauacccuuuguauguucacuugauuaauggcgaauauacagggggagacucucauuugcguaucaaa

SEQ ID NO: 161 - RNA, 75 nt, Homo sapiens; identical to SEQ ID NO: 37

SEQ ID NO: 162 - RNA, 84 nt, Homo sapiens
uugguacuuggagagugguuaucccuguccuguucguuuugcucaugucgaaucguacagggucauccacuuuuucaguaucaa

SEQ ID NO: 163 - RNA, 78 nt, Homo sapiens
auacuugaggagaaauuauccuugguguguucgcuuuauuuaugaugaaucauacaaggacaauuucuuuuugaguau

SEQ ID NO: 164 - RNA, 79 nt, Homo sapiens
gugcuuaaagaauggcuguccguaguauggucucuauauuuaugaugauuaauaucggacaaccauuguuuuaguaucc

SEQ ID NO: 165 - RNA, 91 nt, Homo sapiens
auuuucaucaccuagggaucuuguuaaaaagcagauucugauucagggaccaagauucugcauuuuuagcaaguucucaagugaugcuaau

SEQ ID NO: 166 - RNA, 97 nt, Homo sapiens
aacuaugcaaggauauuugaggagagguuauccguguuauguucgcuucauucaucaugaauaauacaugguuaaccucuuuuugaauaucagacuc

SEQ ID NO: 167 - RNA, 80 nt, Homo sapiens
gguacuugaagagugguuaucccugcuguguucgcuuaauuuaugacgaaucauacagggacauccaguuuuucaguauc

SEQ ID NO: 168 - RNA, 76 nt, Homo sapiens
uacuugaagagaaguuguucgugguggauucgcuuuacuuaugacgaaucauucacggacaacacuuuuuucagua

SEQ ID NO: 169 - RNA, 73 nt, Homo sapiens
cagggugugugacugguugaccagaggggcaugcacuguguucacccugugggccaccuagucaccaacccuc

SEQ ID NO: 170 - RNA, 66 nt, Homo sapiens
gguaagugcgccucgggugagcaugcacuuaauguggguguaugucacucggcucggcccacuacc

SEQ ID NO: 171 - RNA, 73 nt, Homo sapiens
acuuggagagaggcuggccgugaugaauucgauucaucaaagcgagucauacacggcucuccucucuuuuagu

SEQ ID NO: 172 - RNA, 80 nt, Homo sapiens
gcaggaaugcugcgagcagugccaccucaugguacucggagggagguuguccguggugaguucgcauuauuuaaugaugc

SEQ ID NO: 173 - RNA, 84 nt, Homo sapiens
gugguacuugaagauagguuauccguguugccuucgcuuuauuugugacgaaucauacacgguugaccuauuuuucaguaccaa

SEQ ID NO: 174 - RNA, 102 nt, Homo sapiens
cccaagucagguacucgaauggagguuguccaugguguguucauuuuauuuaugaugaguauuacauggccaaucuccuuucgguacucaauucuucuuggg

SEQ ID NO: 175 - RNA, 69 nt, Homo sapiens
uugagcagagguugcccuuggugaauucgcuuuauuuauguugaaucacacaaaggcaacuuuuguuug

SEQ ID NO: 176 - RNA, 84 nt, Homo sapiens
acgucagggaaaggauucugcugucggucccacuccaaaguucacagaauggguggugggcacagaaucuggacucugcuugug

SEQ ID NO: 177 - RNA, 79 nt, Homo sapiens
ugguacucggggagagguuacccgagcaacuuugcaucuggacgacgaauguugcucggugaaccccuuuucgguauca

SEQ ID NO: 178 - RNA, 91 nt, Homo sapiens
cugggguacggggauggauggucgaccaguuggaaaguaauuguuucuaauguacuucaccugguccacuagccguccguauccgcugcag

SEQ ID NO: 179 - RNA, 70 nt, Homo sapiens
uugaagggagaucgaccguguuauauucgcuuuauugacuucgaauaauacaugguugaucuuuucucag

SEQ ID NO: 180 - RNA, 80 nt, Homo sapiens
gguaccugagaagagguugucugugaugaguucgcuuuuauuaaugacgaauauaacacagauggccuguuuucaguacc

SEQ ID NO: 181 - RNA, 78 nt, Homo sapiens
cugaaauagguugccugugagguguucacuuucuauaugaugaauauuauacagucaaccucuuuccgauaucgaauc

SEQ ID NO: 182 - RNA, 23 nt, Homo sapiens
uccaguaccacgugucagggcca

SEQ ID NO: 183 - RNA, 22 nt, Homo sapiens
ugaaggucuacugugugccagg

SEQ ID NO: 184 - RNA, 22 nt, Homo sapiens
uuguacaugguaggcuuucauu

SEQ ID NO: 185 - RNA, 21 nt, Homo sapiens
gaacggcuucauacaggaguu

SEQ ID NO: 186 - RNA, 22 nt, Homo sapiens
cuccuauaugaugccuuucuuc

SEQ ID NO: 187 - RNA, 20 nt, Homo sapiens
accaggaggcugaggccccu

SEQ ID NO: 188 - RNA, 21 nt, Homo sapiens
ugucuugcaggccgucaugca

SEQ ID NO: 189 - RNA, 22 nt, Homo sapiens
caggucgucuugcagggcuucu

SEQ ID NO: 190 - RNA, 22 nt, Homo sapiens
aucaugaugggcuccucggugu

SEQ ID NO: 191 - RNA, 22 nt, Homo sapiens
cugaagcucagagggcucugau

SEQ ID NO: 192 - RNA, 22 nt, Homo sapiens
ucggauccgucugagcuuggcu

SEQ ID NO: 193 - RNA, 23 nt, Homo sapiens
ucuuggaguaggucauugggugg

SEQ ID NO: 194 - RNA, 21 nt, Homo sapiens
cuggauggcuccuccaugucu

SEQ ID NO: 195 - RNA, 23 nt, Homo sapiens
acuccauuuguuuugaugaugga

SEQ ID NO: 196 - RNA, 22 nt, Homo sapiens
caucaucgucucaaaugagucu

SEQ ID NO: 197 - RNA, 22 nt, Homo sapiens
gccugcugggguggaaccuggu

SEQ ID NO: 198 - RNA, 21 nt, Homo sapiens
ugguagacuauggaacguagg

SEQ ID NO: 199 - RNA, 22 nt, Homo sapiens
uauguaacaugguccacuaacu

SEQ ID NO: 200 - RNA, 21 nt, Homo sapiens
uaguagaccguauagcguacg

SEQ ID NO: 201 - RNA, 22 nt, Homo sapiens
uauguaacacgguccacuaacc

SEQ ID NO: 202 - RNA, 22 nt, Homo sapiens
ugguuuaccgucccacauacau

SEQ ID NO: 203 - RNA, 22 nt, Homo sapiens
uaugugggaugguaaaccgcuu

SEQ ID NO: 204 - RNA, 22 nt, Homo sapiens
uauguaauaugguccacaucuu

SEQ ID NO: 205 - RNA, 22 nt, Homo sapiens
ugguugaccauagaacaugcgc

SEQ ID NO: 206 - RNA, 21 nt, Homo sapiens
uaggacacauggucuacuucu

SEQ ID NO: 207 - RNA, 22 nt, Homo sapiens
aggugguccguggcgcguucgc

SEQ ID NO: 208 - RNA, 21 nt, Homo sapiens
cacauuacacggucgaccucu

SEQ ID NO: 209 - RNA, 22 nt, Homo sapiens
uuugugaccugguccacuaacc

SEQ ID NO: 210 - RNA, 22 nt, Homo sapiens
aacacaccugguuaaccucuuu

SEQ ID NO: 211 - RNA, 22 nt, Homo sapiens
ugaaacauacacgggaaaccuc

SEQ ID NO: 212 - RNA, 22 nt, Homo sapiens
aaacauucgcggugcacuucuu

SEQ ID NO: 213 - RNA, 22 nt, Homo sapiens
aaacaaacauggugcacuucuu

SEQ ID NO: 214 - RNA, 21 nt, Homo sapiens
aacauagaggaaauuccacgu

SEQ ID NO: 215 - RNA, 21 nt, Homo sapiens
aucauagaggaaaauccacgu

SEQ ID NO: 216 - RNA, 22 nt, Homo sapiens
uggugggccgcagaacaugugc

SEQ ID NO: 217 - RNA, 22 nt, Homo sapiens
uaugucugcugaccaucaccuu

SEQ ID NO: 218 - RNA, 22 nt, Homo sapiens
aucauagaggaaaauccauguu

SEQ ID NO: 219 - RNA, 21 nt, Homo sapiens
aucauagaggaaaauccacgu

SEQ ID NO: 220 - RNA, 22 nt, Homo sapiens
guagauucuccuucuaugagua

SEQ ID NO: 221 - RNA, 22 nt, Homo sapiens
uauacaagggcagacucucucu

SEQ ID NO: 222 - RNA, 21 nt, Homo sapiens
agaggauacccuuuguauguu

SEQ ID NO: 223 - RNA, 22 nt, Homo sapiens
uauacaagggcaagcucucugu

SEQ ID NO: 224 - RNA, 22 nt, Homo sapiens
aaucguacagggucauccacuu

SEQ ID NO: 225 - RNA, 22 nt, Homo sapiens
ggagaaauuauccuuggugugu

SEQ ID NO: 226 - RNA, 21 nt, Homo sapiens
uuaauaucggacaaccauugu

SEQ ID NO: 227 - RNA, 22 nt, Homo sapiens
auucugcauuuuuagcaaguuc

SEQ ID NO: 228 - RNA, 22 nt, Homo sapiens
auaauacaugguuaaccucuuu

SEQ ID NO: 229 - RNA, 22 nt, Homo sapiens
aaucauacagggacauccaguu

SEQ ID NO: 230 - RNA, 22 nt, Homo sapiens
gaaguuguucgugguggauucg

SEQ ID NO: 231 - RNA, 22 nt, Homo sapiens
ugugacugguugaccagagggg

SEQ ID NO: 232 - RNA, 23 nt, Homo sapiens
ugucacucggcucggcccacuac

SEQ ID NO: 233 - RNA, 22 nt, Homo sapiens
agaggcuggccgugaugaauuc

SEQ ID NO: 234 - RNA, 22 nt, Homo sapiens
gucauacacggcucuccucucu

SEQ ID NO: 235 - RNA, 23 nt, Homo sapiens
agguuguccguggugaguucgca

SEQ ID NO: 236 - RNA, 22 nt, Homo sapiens
uagguuauccguguugccuucg

SEQ ID NO: 237 - RNA, 22 nt, Homo sapiens
aaucauacacgguugaccuauu

SEQ ID NO: 238 - RNA, 22 nt, Homo sapiens
ugaguauuacauggccaaucuc

SEQ ID NO: 239 - RNA, 22 nt, Homo sapiens
aucacacaaaggcaacuuuugu

SEQ ID NO: 240 - RNA, 22 nt, Homo sapiens
agagguugcccuuggugaauuc

SEQ ID NO: 241 - RNA, 22 nt, Homo sapiens
uggugggcacagaaucuggacu

SEQ ID NO: 242 - RNA, 25 nt, Homo sapiens
aaaggauucugcugucggucccacu

SEQ ID NO: 243 - RNA, 23 nt, Homo sapiens
agguuacccgagcaacuuugcau

SEQ ID NO: 244 - RNA, 22 nt, Homo sapiens
gaauguugcucggugaaccccu

SEQ ID NO: 245 - RNA, 23 nt, Homo sapiens
acuucaccugguccacuagccgu

SEQ ID NO: 246 - RNA, 22 nt, Homo sapiens
agaucgaccguguuauauucgc

SEQ ID NO: 247 - RNA, 21 nt, Homo sapiens
aauaauacaugguugaucuuu

SEQ ID NO: 248 - RNA, 21 nt, Homo sapiens
aauauaacacagauggccugu

SEQ ID NO: 249 - RNA, 21 nt, Homo sapiens
aauauuauacagucaaccucu

SEQ ID NO: 250 - RNA, 22 nt, Mus musculus
acucaaacuaugggggcacuuu

SEQ ID NO: 251 - RNA, 24 nt, Mus musculus
aaagugccgccuaguuuuaagccc

SEQ ID NO: 252 - RNA, 22 nt, Mus musculus
caucaaaguggaggcccucucu

SEQ ID NO: 253 - RNA, 22 nt, Mus musculus
aaagugcuuccacuuugugugc

SEQ ID NO: 254 - RNA, 22 nt, Mus musculus
acucaaacugggggcucuuuug

SEQ ID NO: 255 - RNA, 24 nt, Mus musculus
aaagugccgccagguuuugagugu

SEQ ID NO: 256 - RNA, 22 nt, Mus musculus
agugccgcagaguuuguagugu

SEQ ID NO: 257 - RNA, 22 nt, Mus musculus
acucaaacugugugacauuuug

SEQ ID NO: 258 - RNA, 22 nt, Mus musculus
aaagugcuucccuuuugugugu

SEQ ID NO: 259 - RNA, 22 nt, Mus musculus
acucaaaauggaggcccuaucu

SEQ ID NO: 260 - RNA, 23 nt, Mus musculus
aaagugcuacuacuuuugagucu

SEQ ID NO: 261 - RNA, 22 nt, Mus musculus
acucaaauguggggcacacuuc

SEQ ID NO: 262 - RNA, 23 nt, Homo sapiens
acuuaaacguggauguacuugcu

SEQ ID NO: 263 - RNA, 22 nt, Homo sapiens
aauugcacuuuagcaaugguga

SEQ ID NO: 264 - RNA, 23 nt, Homo sapiens
uaagugcuuccauguuucagugg

SEQ ID NO: 265 - RNA, 23 nt, Homo sapiens
uaagugcuuccauguuugagugu

SEQ ID NO: 266 - RNA, 22 nt, Homo sapiens
uuuaacauggggguaccugcug

SEQ ID NO: 267 - RNA, 22 nt, Homo sapiens
acuuuaacauggaagugcuuuc

SEQ ID NO: 268 - RNA, 23 nt, Homo sapiens
uaagugcuuccauguuuugguga

SEQ ID NO: 269 - RNA, 23 nt, Homo sapiens
uaagugcuuccauguuuuaguag

SEQ ID NO: 270 - DNA, 11439 bp, Mus musculus
ggaaagccaggttgtctaccccacagagcgcttctgaagaccaaactacataactcacaagaaagtgccttgtaaatcgcccggaaattgcaaaaaaaaaatccttaattagccaatgaggctgtccctactgcccagcggccagccctagtctctcacaacagtgagcacccagtaggtgctgggtgtctttgtgttcaacaaataatttccagaattcaaacaaacaaacaaacaaacaaacaacattcattcaacaaacattttctgagacactgaccatgtgcccagtgcaccaggctatggctgaggtacaaagacaatgagaacagtctctttttggaggctaattagtactctcatttctaccagcactttctttggccttttaaaccaaccatttttgtccagacaagaccaaaaaaaaaaaaaaaacaaaaacaaaaaacaaaacaaaacaaaaaacccaaaaaaacaaaaaacaaaacaactgggttgctttagggaagcccggcttggagtggacaatggtgtccaggcccctgaaaggggctgattggattatgaggcaaatggaggcagaaggagcacgcaggagacaaatgcagtgggcggagacccgctctgagttaatcaggctgattgaagggaccttttctgtcttgccgagtggcccgggggggctctcactagtgcactgctgtgtgcacatggagactggagctaccctggtccctctctggcaactgttcattcatttgatgctcacaactccctgtcctgacccagggccggcctgccatcacgcagggaagcagaggtcgccaagcggtttccgacggggcccccccacataacgttagcctcgcggtctcttcggttttagcagcagtggacatgaacctgtttccagcctgcatcctccagttcctctcgggactcctggctccactgtgctcacctggagccccgccagcccgcctccccctgtcctccttcctcgccactccagtgtctgtctttccttcacagccccggcttctcgaggcctgtctacactcgctgctttccttcctcacctccaatttcccctccaacccactgcttcctgactcgctcttctccatcgaacggctctcgctcaggcctgtcgcgtcttcctgtgccatttgctgttgtgctcaggttccacgagctgcccatctccacagaagagcagctggcattgcccaccggccatgccggctgaagaaaagaagactgaggaccccaggatgcccagcgcgaggaccccaggaagcccagcgcgaggactccacccacgacgcccagcgcgaggacttcacgcacaacacgttgcaaccctcctggattaggccaaagccatcatctggaatcctgcgtgggaccctggacacacggacacagacacctgcccccaggaccctccaactgtaaatcgattggaagcgcccttgtgagctggggccttcaaagggctgaggagaaaatgcaggccgagagaccagcttctcaaataggcttcaagcctccatgacctgagctcaccccctggaaatgtctggaaaacataatgggcaggttttctgtcttcaaagtttccatcaaaacctcttcaagttctttattgtttaggactgagacacttggggacagtaatggggactttcttttgtatctgtcttgaaaggccaaaatatttttatattgctgtagacaaagccacctatttacaaatggactcttgttccgtcgtttcccaccaggaagaccgtactatgtttgtgtctctatgtattctggggtcttgaaacaggtttctcatggggatggccattcactagagcccagaggggcagaaggggaagtgtctaccctgccaagggggtctggggaaggaggggggctagtctcagatttgctccttgtatgtctagggtaaaccctgggagaatctggtggtaaaagaacatatgctgtgtttgccccacatctgccatttgtgtgtgtttctgggctacgggtttgcacactttggtggttaacatggtgggaggaagtaagaagggccaagagggcacgggtgctctggttttcctgctgggaggctgtttcactggtgtgtgttacatcacataatgcatgtttagctgggtgagtgacaacagtatgaagggacagagaaacaaataggtgtagaaagagagtcacagagcacacagtcaccattctgtgcccaggcgttcattaaatgttcaatagaatgctgagctctgatagacagagcgggccatggacacactggtattgggtcatcatggacctaaggagtttttttaattgaagaaggaatcttacataacagaagaaagaataagaagagctgtcaaaacgagctctgaggtttttgaagccccttaatataaacagacagacaatggcgtgatcaggaaagggagaaaacaaacaaacaaacaaacaagatgcttacagaaatacgagaggacgtgtgagcatgcaaagagaacccagctaggttagtatcggggtgctggtggatcgttaatcctgtctgggtaccccagctccaaggggatgagaggcagcaagtgagaggacacataggaaactgtaacaagacagaggcgagcagatggggagcttcagcggagtgttaagctaggcttcgcaatctgaccacttaactctcccagcaaagaaaagaaaggcttaaatactaaaaagaaggaaagctggaagaaaggatggtagatgtgaaaggcatagaggatagaaagcaatagggagtggagtgcaggtgctgtggagagacgcttgcaggtgactcaattcaccagcctgtgcagctgagaaagacagccagtgacaaaggagaagaagaaaggtaacaatacactcacaaataggtagatgaatgtagctggaagacggggcctggctagggacgggatgagaggctgtgtcagcagactggttaattcccatctgtgcttctgagatggacaatgacccacccgaccaagagacctttcaggcatcaatatgtcagggagggcttcaatggggcaatggaggcgaggcccattaatagcagattatgatgtatccaattaggtgcacctgccctctggaagtgcagaaaccagtattgaaatggaaagaaagactgaatggaagtccgagagtacaaatgaatggatgggaaaaaagatttgcattcgctcttagggggagggtgctgctgagccccagggtcatctgatctcccggagaataaaagggcgagggagtgatagagagtaacaataagatggagaagaaatgaaaaggagtacagagagaataagtggggacaatgaagtctaggagcgcggcttgtttaacacaagtcatccatattaattcaaaattgaggccctgggatgggatgggctgtgtccaagggatgaagctgttgcctagcatgtgccaagttctgggttccatctccgcctggcaagacaaacacaggaaacaaaattatagcccagaggttgtaaatgttgtttttgatggttttgggacaaagagctagatcggttggaagatagagaaataggaagaggtgggaaagagttagatgagcacaagaggaagcagcagttcttaagataagtgtttaaatatggtgaatgcgtagctcttgggtgtgtcaagatgaaaataacgaaaagaaacaagtttagggatgacggcacagggatgagcggaagcggtgactttagttaatcaccgtggccaactcaagccctgtcagggatttaaaaaaaggtacaaaggcgataaaggaagacacatgcaatacggaagagagagacaggacagacagacacagcgaaaaggctgaggacatgcctgagagggtcgcgaagggatgagagagagaacagcgagaattctgcttaattcaaaaccgggctcctgagatagctaaatatctcctgagcccaaacaccagcacaggagattaaataacaaaagagctgcaggcagacccaagtggagaaggcttgggaccctcagatcaggcttcaactaaatctgtgcacctgaggtccataaataaaagaataaatattgaaataaaggagtgacagaaaggatgaatgagcacacatgactgaattagaaatggaaatggctagagcagccagtaaggagtggcccatgggacttctgctcatcttattctgggcacctgaggtcccttcgtgagaagatgcagggagaagaggggaagggaaaggcatgagagcaaatgaaagagaggcgaggagaggaaatgagaaagtggcagagaggggaagcgatcggtgggtgcctcggaagaggaatgtccacgaggattaataacgaggctctcattaatcctaaagatcgatgaggtaatccagacccaggttcttaaggggaaggagaatggagagaattaaaaaaaaaagacgcaaaaaaaggagagaaagataataaaacaagcaacatgatgaatgggtaattaagagaggctgtctggacagaaatagtaatgagttcccatgcatagtccttaaatagataaatacattatacctttcaaagaatgcgtgaaaaatcgaagaacaccatggcccccaaacaaggctgcaccttccattgctgtaataaaatacatgcagcggagagccataaataacggaaaatacttaaatgtaaaaggtgaccagacagagggagaagattgcaagcccagatacacgagggagggaggcgatgtttgctttgcatgtgtaatacaattatgttcacaggaaattaatagatttaaaaaaaaaatccaaactaagactgggagaaaaaacaaggaagagagggaagcgagatctgagagatggggctgagactcccggggtaggctcagcatggttttgactattctgcacatccggacttagaaaagtgagtttgtcagggcaggaagagaaaggtgaaatctggagcgggcagagctgggcagatggatagaaaaggaatggaaactacacaggttgtgtgtgtgtgtgggggggggaagtgggggctctaagatctgaggttaggcttgtttaaagctgagccattgttgtccccagaggaatcaaatcttactgcttttgtgaaggaagagaggatggcaacgatattgcaggaaaagagggaagggggtaggcaaggaggcagggaggaactttcatgtgctattcgctgactgggattggtaatagatagctcttggcccaccacaagctgcttccacacagcctaactgactgggagcagctatggatcaccatggccatgttttgcttgcagggggccatttcactgtctgtcacatcttcagtccatagacacatccctgcacagtgccctttccatagacgtggataggagggaagggctcaggcctggggctgtggagatgattgcatgaatagggggcgctatgcccagtgggtaccttgtctcctctctgggaaaggatctcttctgatcctgatgggaaggggcttagccagccaaagggcagggataggaggctttgtctgctgcctgctggctaccatcttgagtgtgacttacaccagggtgcagacaaagtgagcttgacctggaagcctagaggcccaagaggcccgggtgaagcaagtatctctgagatcaagcctacccttcagctcagagagtggacccctgcctaggctcccacacatgatcctaggtcccagatatgccacccctggagcccgtttcaaggcccatcttccagaatgtttaaacctgtgtatgtaaccagtaaaatgggagtttgtactgtgtattgaatctgtcttcttattggcctctgtgggctgctgggggagacctggagagttgtttattctagtgtcccccaccccctgggactctttgcttttgcccagttttctttctctgttgtggataagattttgtagagttgtgtgttgtatgcttgtgtttgaccattaaaaaaaaaaaaaaagtcacagccctccttgatttctcaattcctattgggggtgctaaattccaaaccaaaccaatccaaaccagattcatgtcaagtgcccaacaggaggataatagcttggctggatcaagggagcatgtgcagacccaagaacagagtggacagatgggctataccgcaagggggctttgagagctgagtgcaggctaagtagagagaaggtatggaaggttggggagggtggggctgctgctacctgagttgctgagggtctgagacaccagcaaggaggaggggcccctggaagaagcgttttgagatgactgaagatggaagatgggcgctccctacgcttctagcttcaaggtttttagtgtacgccagccgcctccgatcgtggaagacacagcctaaccttgtcagaagccaccttctgtgcccccagcaccacgtgtctgggccacgtgagcaacgccacgtgggcctgacgtggagctggggccgcaggggtctgatggcctcctgtttcagaatctggggcgtttatcatccttgggacaactgctgacctggctccggaagattgtccacacaagtctgagtgacggagtgagatgagggggcgggtggagatgctcagaagaaaacagcctttgcatggggttcatttccagctgtttagagagtcagccaaggtgacaaggtggggttggatgcggagaagaatgctgggactacatgtgggctacagctgtgaagtcagtgacatggggcagcatggggatgggtctctaggtgggccttgtctcaacattcatgccagaagattggaaggggtaccatgggttactgtgggtcagtgggaccttggaaggaacaagggcttgggtaggtgactttgccatgtcaccttgggatctagttgttttgagtctttgactcttaatctgatcttgaataacaaatgtctctagttctggggttctgagagtctgtggtaggattccctaggattcgtgtggggagggcggtctgcatgtgctcccaagttggtgtcccagcccttctgcctgctcagggctgtcttagacatatttgcttttcctttcaacatcataaaagaaggcaaattttgtcttttgattgacactcatatgcaaaccacaacatcctttctctgataatactgggacctacagctctcctcggctccccccgctatttgagcccacctttggggcaggtaggccaaaggccctggtgttttctgaggtgaagttgttaatgtaaagatgtgtaactcagagcagggggaagaaacacaccctcaactctgcttccccgtgctccatcttcctttctgccttccaaattcttatgaacaccgtttttttttttaaatttttttccatttaatgagcatccttttttttttttttaaagctccattggtggtgcttacttttctgttggtgttttgtgtctgttgcaatgagcatcttgctgtccacgttttcgttgctccatggtctctctcacctgcttgagttccattgaatggctatgacataatgatctacccatcttactgttgctgatgcatgatcatctcctgtcctgttttttttttttattttacattaccgatgacaccataactaatggctttgcacaaccagctttcccagcatccctagcgccccctacatctagctcttcccatggttcctttcttttctccttgcttccttctgctagactgtgagcccactcagggcaaggagcttgtggaactgcgctctgtatttcttgtaggatgtctatgaatgttgactgcgtgtgtgtgtgtgtgtgtgtatgtgtgtgtgtgtgtgtgtgtgtgtgtgcgtgtgtgcgtgtgtgcacgtgtgcgtgtgtgcgtgtgtgcatgtgtgcgtgtgcgtgtgtgtgtgtgtaagggtggacaggtatgagtgatggagtgattgaaagattggcagacagatgagaggctagatgaatggatgagtgaatgaatgaatgaatgagtgagtgaattaatggatgggtatattctctggttgagcttcttggatgaaagggtaatgtttgaatggctgttggttgattgaagaaagcctacagtattttatcttgtctgataatgtattagcatttaagctgatgtattagtatttaaggtgtctgctgactttcctcacctctctgttaaaaaatgcttttgtgtgtgtaaaacttgttctaattactaatgaaggctgaatacttttcacccatgtttgttaacaaattgtatcttatcttctgtgaactgtttgtccaagtccagtggctcatggctctttcaggctcttggttgagggtgtccttagactaggcaatggcctttcacagatatctacagaatgatgtgagaggctcagctgacttctagctcggcattagaatttacttaccctgggaaacagggaggctgatatcttgcaccagcaggtagaaaacacttttaggcttgtgtctgggttcaccatgcatttctagcttgtgagccctgacctgtgctctgtacctcttgtggtgtggcagtcctccaccttgtcctcctgagtgatagactacatatatgcctctcccctgttctggggtgtagtctctgaggccatgtcttagaggaactggcttcttagtgctctgtctagacatccccttagctctagtcttcccatgcacctcagtggcaggtgtggtcttaatttagggaatgaatgagaaattcagttcaggactgatgtgaccctcaggttagttctaggagcaaggccattttatggtctcggctcttcccaacctcctcacagggcagttatagccatctgggtagggtgtgtgttccgggagagctcatcctgccatccagaactccctcccacactctattatagcacttagcgtgtctgcctgtgttcagccctctcaccccatgcttatctggacattgaagcttggaaagccagtggtgacttcaactgacttttaattcatccacccatccatctggctatccatctagccatctgtcagtcaatccatccatccatccatccatccattcatccatccatccatgcatacacacattgggcctccatcacttgacctggtgctttgggcctctctccctctcctgaaagcaaaccccttcaccttgggcaagctgtgtgataccatgggctgtggtcatgaagcaaggcccccattcacagcctcctcctcctcctcctaggtcacggctctgagcacgtcccagctggacccctatcaccacagctttttctccataaggcttctcacacctcacaccctcctcctgtgatccaggagggccagattcccagagtgccctggggctggcccttcccaccatccgcggagctgcctccccaggcttcacactgcctggtgcatggtccctgtcatgagcttggccttcacctttgcagcttcctccaccagatggcagtggttgtgactcgtgtgcccattccttcgcaccttcattcattcattctcttacagccagcaagcatctctttaaaaaaaaaaaaattagctagtgctatactagagccggctcacaagcacccattgcaccactgtcctttgaaagaaaagatggggacatttgtaaagaagctgtgtttgtgtggccaataagaggagctccttgatttcaattcaaacttcaggtctgagttctattgccagcttgattgctctctccaattttgttcctcacaaaactaggttgattagtaatcagcaaactgtgttccctgggtgctcagaaatcatagcccagatccttaagaggcccaaatggggagtggactaagcacgaagcagctgcctctggctgcgagcaggctttcaaggctgtgagacactctgttcttctatgaggatgatctgagagccgttctggccactttggagctcagctaatgcttggctcctttttagagaaaattgcaggagatggatgctcgtacaagtgacaaggatcatgtcccaatgtcctttctgaataatccattcgcagacccaggcgagcaaggcacatggcactttgcaaacccctgtgtgtttcaggaggacccatgctaggggaggcctcctttcagctttctgctgtgaaggggaagagacgcagggctcttctgagttctcctcccactagacgcagtaggggcggcaaggccagaacctggagctggacaaattgtgtgatggatcttgcctcagggctgttgtgaggattggtataactggacacttctgactgtgaccctaaaaggcagtgtatgtgtccctgggggtgtgccagagcattggaaaccctagccctggagtgggggtggccttgcttgggtataatggggaatactttgtatctgttaaatttgtatattttcctatagagactttaacaccacgggataggggactctttagatagtacctgtgccctccgcccccctcggacacatccacacctacctctaggagaggttggggttgtctttagctctctgactgaggaccaagcctctgactcagaactgtatatggcacctagttacatccctttccaaaaggctcttcccaggggagcactcggcccgatctggcagaccccattgtccctttcccaatgtcctctctcccactaacagcatcaggccaaactgcctgagatctggtgcctaccacagtgcctggccaggggtagggcttcagtgaccttctgttgtatttgtgtaggtagatgagtagctaacaattgtaacaggtcctaggggcagatgtgtatggtctcattcagtggtttgtaatggagaatgtatctgaacccatatcaagccatctctcttccttaacatgttaagcagccactgctgtcctgggttgttttgcaccttgccttgttgtctttcatgctaagtaaaaaaaagtcctgctttctgctgagagtctgtttcttctggaactcacctaggtgtgatctggcagggctgctaagctgtgtctctgcccctctgacccgtagtcttcttctctctcaggtgtgcgtcactggcaagcctacctcacagggttgttgtgagggctaagggctgatcggctgtaacagtgtgatgtccatgctggctgtcaccagggatggtgccatgatggggttggcttgaaatcacaacctataccgttattagtggtcactgaataaagagtctcactgtggacactgaggttgtaaaaaaaaaaaaaaaaaa

SEQ ID NO: 271 - DNA, 1317 bp, Mus musculus
ctcagaggatctgtcaaagtaccttagatttgccctaatggacataagcagcagtgggcgcagaaaccttgctctgaagcctctctggttccaacatctgcggaagagtgcttgtgtgtcaccttcagctggcatctccataacaccaaaattgaagtgtgagaagaagaagacccaatgcccggggagaagtacggtgagcctgtcattattcagagaggctagatcctctgtgttgagaaggatcatgatgggctcctcggtgttctccaggtagcggcaccacaccatgaaggcagcccggattggaaggatcctcatctccactcgagggtactccacctccattgtagagaggggtcttgaatagaaagcacaggtagatttcttgccagtttcgtcgtcggtttggaccagggaggcagacaggaatgacccagtgatgtctgtttccaagtagaatgggttctgaggcttagggtgatagagaacgggcgacttgcggaaagccctcttcagggattccaaggcctcctgctcctcttctccccagtagtagggctctgaactcagcagttgtctcactaggggtgctgcgatgacagcgaagttctccacgaagtggcgatagggatagacaaggtcaatcacactttgaagacacctcctgctgccagggacagggcaccccacgatgaggttcataaggttcttgttcagtttcacccctttgggggatatgttgaagcccaagatttcagcggtctggcgatggaactgagttttgtccagtgaacagtagatgttgtgataccgaaagcggaccaggacttggcggacatgctgggagtgttcctcctggctcattgagtagaccaggacctctctgccatggcaaatcacaaacaaccctaggatgtcttttaggatgaagtgaacaatgttgttcccttcgtcagagtatgagttcattgtgaagggccggtagcatctcatctggtgaaggccgaaaccgaacgatgctctccaagtgtcttccgtgtgtgtgaccgtgtgcctcatctcactttccttaatcccaagcagctccagttttgtgaaccatgccgctccatgtagctggtcgaatagctctggtaccatctgcgtgtagtcttgtctgtcagtcagcatgtcatacagtatccagtattcctcttgctccctggctttctcttgctgcctggctttctcttgcatcctggcgctcacaggttccaaagtctctcgggccccagactcgtaaaagacgccgctttgatcactgtctccagcctg

SEQ ID NO: 272 - DNA, 3584 bp, Mus musculus
tcactgcccctcagggggcagggggctggtgattcctgagggagccctgcccccttccagggtgaattttcctaagtcaccatggagaaccagcagcccagaatcagtgccctgccaggcagaattgcttggccataacaaagatccattgcctttctgatgacacccagcacctcggattctgtatacacgggaggtgatttaagcatcttactgccaccctgaaatctggggtcctttatcctggcagcatctagacccgtaacgcccacttcagaatgggagggaggatgtctgctgccctgtcgtctccacacccgaggaatgtccgtgtgtgtgtgtgtgtggtgtgcacagcgggctgcacacagcgggctccacggtgctcgaatggaggccctaatgtgaatggagactagcctgctggtagagaccttggcagtgaccgctgtgggcaatgagccatgggccaacaatattcaatgctgtgtacctgatggatggttactgtctgagactgaggctttttgggccaggagtaaaaggacttggtttgtacttgattgcgtcctgtggatgcttgcctaccaactgactcatcctgacgtgccaaaggtcctggctttcccctgtgtgggtaagggaagaagagagttctaatgaagactatagcagactgaattactctctgagatgaggactttgctattgccattgcagaaccactggtggaagttgttgttaaacagtgggtcttgcatgcaagctctacagttatgcacagtatttccatgacatcccaagccatgaggttcatgctgccatatccccctgtttaacaagtgtggaaactgataatggactcacaattgctgatgtataggttctaatgttttctattgtggacacgggacacccagactgagaaccaatggctccagtctgatttaaacatgggtccgtgctcacacactcaatacctaccctctgtgctcacaagtcctgtgtgagtaagactccaaattattctgtcttgttttactcaatggtatgctctaccttattatagagtataccatgtattgagacaagaatgccagtcattctgagaaacaaaatgcaaccattagttgtcattttaattagtgacgaatagttgtgtcaatcatcaaacatgcttgtataattggatgagaagaggtgtaacacgggccaatgatgacgaggtttcgggattataagtcacggacgatgactacgtcactagaggtctgaggtccatagcagaagatgcctcatgtgaccctctcatgcaggggttgcatgtcaggcatgtgcatacagtagatattagccatgcactctcaccatttcatagtagatcacagaatcaagatacaaatctccatgcacggaaaggatttaaggcattgatcaatcctgaacataaatatttggccatcaatgatatttacgcacacactgaaaaatctattcagaatgacttacacaaaggtttatataacctttatcaaaggtaatttatatagtaagattcatggctgaagtcttcatgatggtcaaggccttaatttttaaaattatcaatttttttcttttctttttttttaacttgaatttttttttaatgaaaactatcatgacaatttgtctcttaaatgaaaggagaaatgtaaaactaaatggacaaatatcaacctttttattatctttgctgttgtgccctccctggatgggtgctggcacatgatgtcttgcctgctggtccgtggttgcactctcaccatgtggatggagctcggcagctgaaaatgcattcttccaccaagatgatttacacagcctagctggatcaggagccccctaataagctgcttcagattgtaatatagacacagtaatgtatgcctgcaggatgagtcccaccttcttcattagtcttgaggtttgagattccctgtttgtgtctgcaggattcagatagtaatggatatcctgcaagtcggcttcgtagagtctcccttgaaagtggacctcatgatctagcctttatgtccgtggattgacactgtgatggattccccccaagttcatttagatattctctcttattcataaatgtcacggtcagctctgttcttatagataccagaatggatacccctaagctgatgtatatacacccttcctcggttgtgagattcctgctctggtggttgtagtcataagatatacatcgttgcgtacaccttggttgtgggcacgcacactactgttgaattaccaccttgtccaagtggatggatgccctcaatgggattggcactttctccattgctcctgcactctgttatcagcacttcgggttcataatgtacacacagcaatggattttctgcagctgattttctatattctgttttcttcgggtaagcagaaacagatgccttcctcactggtcaccaggttcaaggtccctcatatgtgtcaggtgcatagtcaccaacaaaggccaacttcacatcaatgggtggatcgtacctcggcctaagctccaggattgattgtgctgttagagtcacagctacaaccggctcagggaacccagtggaagaggaggtggagtgtgagagccaattggaatggaagacagtgaggaaacaaggccttcgagacacaagaggactgctgagctcacaaagactgaggcagcaccctcaggacctgcacaggtctaagcttgatgcgatctgagtactgagagggcaactggacacaattctatattgataacccagaagctttctccagttgataaactctttcaaaggaaaaacttgttttctaccatggagtgacactggatatagaaactaccgttaacagcagactccatggctcagacttccaatacaatatgaactcagttgcaattttttgcaaggtttttcgactcatagttctttgtctgggtactttttcaccctacagagaatttctgtatatattatcgtttctgattctagtgttttgaattttcgctgggtctaaatatctctgtctcagtgtcagtatgtgtttctcatgatttttctttgcttctctttttcctattctttgcttgtattccctggtttgtgtttgagtgtctgggaactctgagggtctggttggtgatattgttgttcttcctatggggttgaaaaccccttcagctccttcagcctttcactaagtcctccattattatgattcctatgctcagtccagtggttggatgtgagcccccccccccctctgcatttgtcaggctccggcagagcctctaaggagatagctatattaggctcttgtcagctttgtttgtttttattctgtcttctctatactgtttattttagataactgtgtttattctaatgtatgaatgaataaatgggtgtgtgtttgggtgggtggggaattggggagggtgggggtggatgtgggaggggaaactgtaacacaaatacattgtataaaagtacattttaataaatgtatttcttctatgactaaaaaaa

SEQ ID NO: 273 - DNA, 1315 bp, Mus musculus
ctctgcctcagaggtcttctcaagccactactgttcccaaggatgcctaattgcaatgccagcttctggggtgaacatggacccactgtcatcgggcaaggtctaggatggacatggagacccaccatctgggagattccagagtggacgtgaacccaccatcatcagagagcttcatggagtattcagaagctggcgggtgtcacctggaggatgtgactgggtaccagtgccagagcacgtgcccactgagcagtcttcttcactttcctcgatcctcatgaaacattccgggatctttacctcaaaggactccagaggctacatcattgtcttggaactggtcagtggctggagctctctccaggacaagtgcttggaggaagaacctggacaagcttccctccccatactgttgactggatcttgtagctaccgttagccaccatcatcgccattgtgtgcctcttaggagcatttccaggaggtgatgccagctggggaaagagcctctgtcccacagccgtcggtgcccgctgcagcctgcgcccaggatgtcacagcctcagctcaacacctctgctggacagcttcaggtggtgattccagctccagggttgctgcggtcaacactgggtacttgaggagaggttgtctgtgatgagttcgctttattaatgacgaatataacacagatggcctgttttcaataccacctgccactccggtcactcggcagtacataccaggtgtcacaccactgcccggcaggtgacaacgctgaattggtcctcaggagcggatgttcaaggaaccctgcctatgcaccatgctagctgccagtcctaggttgggacattccactggggacatccatgggagagctatggccattaacaagtcctgagccctgacctgaagccatcagtggcctagctgctctacctgactttgtggcctcccttcctggatctctcgcttacgacaaccgacaacaaagatgtttgactccagaagatgctccccgcggcccacctgagaggactgtccacaccccaccccactgcgccctgtgccatctacctctgagtccccaacttctcacccatgtttcaactcccaccccagagccttgtattccagatctgagcagcccttgcttaggaacctgaggtggggggtgggtaggcagacccaggggactgagcagggaggggaaggaggggtttctgctcttggctttcaaagttctcagtgtgtgtagccctttgaataataaacatatgtcatataatgtaaaaaaaaaaaaaaaaaa

SEQ ID NO: 274 - DNA, 1595 bp, Homo sapiens
agcccctagcgcagacggcggagagcagagagggagcgcgccttggctcgctggccttggcggcggctcctcaggagagctggggcgcccacgagaggatccctcacccgggtctctcctcagggatgacatcatccgtccacctccttgtcttcaaggaccacctcctctccatgctgagctgctgccaaggggcctgctgcccatctacacctcacgagggcactaggagcacggtttcctggatcccaccaacatacaaagcagccactcactgacccccaggaccaggatggcaaaggatgaagaggaccggaactgaccagccagctgtccctcttacctaaagacttaaaccaatgccctagtgagggggcattgggcattaagccctgacctttgctatgctcatactttgactctatgagtactttcctataagtctttgcttgtgttcacctgctagcaaactggagtgtttccctccccaagggggtgtcagtctttgtcgactgactctgtcatcacccttatgatgtcctgaatggaaggatccctttgggaaattctcaggagggggacctgggccaagggcttggccagcatcctgctggcaactccaaggccctgggtgggcttctggaatgagcatgctactgaatcaccaaaggcacgcccgacctctctgaagatcttcctatccttttctgggggaatggggtcgatgagagcaacctcctagggttgttgtgagaattaaatgagataaaagaggcctcaggcaggatctggcatagaggaggtgatcagcaaatgtttgttgaaaaggtttgacaggtcagtcccttcccacccctcttgcttgtcttacttgtcttatttattctccaacagcactccaggcagcccttgtccacgggctctccttgcatcagccaagcttcttgaaaggcctgtctacacttgctgtcttccttcctcacctccaatttcctcttcaacccactgcttcctgactcgctctactccgtggaagcacgctcacaaaggcacgtgggccgtggcccggctgggtcggctgaagaactgcggatggaagctgcggaagaggccctgatggggcccaccatcccggacccaagtcttcttcctggcgggcctctcgtctccttcctggtttgggcggaagccatcacctggatgcctacgtgggaagggacctcgaatgtgggaccccagcccctctccagctcgaaatccctccacagccacggggacaccctgcacctattcccacgggacaggctggacccagagactctggacccggggcctccccttgagtagagacccgccctctgactgatggacgccgctgacctggggtcagacccgtgggctggacccctgcccaccccgcaggaaccctgaggcctaggggagctgttgagccttcagtgtctgcatgtgggaagtgggctccttcacctacctcacagggctgttgtgaggggcgctgtgatgcggttccaaagcacagggcttggcgcaccccactgtgctctcaataaatgtgtttcctgtcttaacaaaaa

SEQ ID NO: 275 - DNA, 498 bp, Homo sapiens
ggtctgaaaaatgatattcattgtcctaatgtgtaaatttcgacaatttgcaaatttgtagattctttagaatagaactaactcaagcccttcattctgcagctgaggctcactgcccccagtgggcagtgggtccagggggtttctgaggacagggcatgacccagccctgctgcccccaagatggcacctggcttggaggggtgaggggccctgttagtctgactttgaagaagaccagccttccagactcgcttggtgccctgacaggagccctgggctcccccagtgttgcctgggtctgactttgcctcagtgaaaactgcctcgaattctttcttgcaccgatgggcagatgggcagtgtcggaggatcgtgtcatctgtcccgtggcgctggttggcttggtcaagtcagtgttcaaactatctcctgctctttcaaggggatctggggctctagaagattagaggacttggagaggttagtgacttgctc